(19) United States
(12) Patent Application Publication
(10) Pub. No.: US 2022/0062383 A1
(43) Pub. Date: Mar. 3, 2022

(54) REGULATION OF A FOREIGN BODY RESPONSE

(71) Applicant: THE JOHNS HOPKINS UNIVERSITY, Baltimore, MD (US)

(72) Inventors: Jennifer ELISSEEFF, Baltimore, MD (US); Drew M. PARDOLL, Brookeville, MD (US); Franck HOUSSEAU, Ashburn, VA (US); Liam CHUNG, Baltimore, MD (US)

(21) Appl. No.: 17/423,043

(22) PCT Filed: Jan. 15, 2020
(86) PCT No.: PCT/US2020/013748

§ 371 Date: Jul. 14, 2021

Related U.S. Application Data
(60) Provisional application No. 62/792,887, filed on Jan. 15, 2019.

(51) Int. Cl. A61K 38/21; A61K 31/506; A61K 31/352; A61K 31/635; A61K 31/45; A61K 31/4725; A61K 38/20; A61P 19/04; A61K 45/06

(52) U.S. Cl. CPC A61K 38/217; A61K 31/506; A61K 31/352; A61K 31/635; A61K 31/45; A61K 45/06; A61K 38/2026; A61K 38/208; A61K 38/20; A61P 19/04; A61K 31/4725

(57) ABSTRACT

IL-17-producing γδ T cells and CD4⁺T_{H}17 cells were identified in fibrotic tissue surrounding human breast implants. In both murine and human tissue samples, senescent cells developed around the implants, which was linked to the IL-17 response. Activation of the T_{H}17 pathway in the foreign body response/reaction (FBR) defines an adaptive immune response to synthetic materials, providing multiple new targets for therapeutic inhibition.

### REFERENCE TO RELATED APPLICATIONS

This application claims the benefit of U.S. Provisional Patent Application No. 62/792,887, filed on Jan. 15, 2019, and is hereby incorporated by reference for all purposes as if fully set forth herein.

### FIELD OF THE INVENTION

Embodiments of the invention are directed to compositions for inhibiting foreign body responses (FBR). In particular, compositions comprise interleukin-17 inhibitory agents.

### BACKGROUND

Synthetic biomaterials serve as the building blocks of medical devices and implants. Biomaterials were historically selected based on their physical properties such as mechanical strength and durability while at the same time inciting minimal host response after implantation. Despite that many advances that medical implants bring to medicine, synthetic materials suffer to varying extents from the foreign body response (FBR) that leads to a capsule of fibrous tissue surrounding the implant (1). Manipulating chemistry and surface properties can mitigate the FBR to a degree, but even a minor response can lead to device failure over time which necessitates surgical removal. While fibrosis may be leveraged to stabilize some implants such as orthopedic implants or stents, it can also lead to implant contraction in the case of hernia meshes and breast implants. Silicone breast implants are widely used in medical practice but develop fibrotic capsules that can necessitate replacement (2). Further, some recipients experience breast implant syndrome that includes increased risk of rheumatologic disorders (3). Recent reports on lymphomas arising around synthetic breast implants designed with a surface to enhance fibrotic immobilization further validate the relevance of murine studies demonstrating the pro- carcinogenic potential of the FBR (4-6).

The classic FBR to synthetic materials was first defined in the 1970s (7-9). It is characterized by protein adsorption and complement activation followed by migration of pro-inflammatory innate immune system cells, in particular, neutrophils and macrophages.

Macrophages fuse to form foreign body giant cells and fibroblasts are activated to secrete extracellular matrix leading to formation of fibrous capsule. Macrophages and the innate immune response are considered central to the FBR and implant fibrosis, however, since the innate and adaptive immune systems are intimately connected, it is possible that the adaptive immune system is also contributing to the FBR (10). Implantation of a biomaterial or clinical devices may therefore impact immune memory and systemic immune responses with yet unexplored clinical consequences.

### SUMMARY

We now provide compositions for the treatment or prevention of foreign body responses (FBR) and/or the treatment or prevention of such immune responses. Methods of preventing or inhibiting an FBR include administration of compositions comprising an interleukin-17 (IL-17) inhibitor.

In certain embodiments, a method of preventing or inhibiting a foreign body response (FBR) in a subject, comprises administering to the subject a pharmaceutical composition comprising a therapeutically effective amount of an agent which inhibits interleukin-17 (IL-17) activity or function. In certain embodiments, the agent inhibiting IL-17 inhibits IL-17-producing γδ T cells and CD4⁺T_{H}17 cells in tissue surrounding the foreign body. In certain embodiments, the administration of the agent inhibiting IL-17 results in reduction of expression of p16, p21, IL-17, type I collagen, S100a4 or combinations thereof. In certain embodiments, the method further comprises administering a senolytic agent, a senomorphic agent, an inhibitor of interleukin-6 (IL-6), an inhibitor of interleukin 1β (IL-1β), an inhibitor of tumor necrosis factor α (TNFα), an inhibitor of interleukin-21 (IL-21), an inhibitor of interleukin-23 (IL-23) or combinations thereof. The senolytic agent selectively lyses or selectively kills senescent cells. In certain embodiments, the agent inhibiting IL-17 expression or function and the senolytic agent are administered concomitantly or at different times.

In certain embodiments, the senolytic agent or the agent inhibiting IL-17 comprise: antibodies, antibody fragments, oligonucleotides, polynucleotides, antisense oligonucleotides, enzymes, gene editing agents, nucleases, peptides, polypeptides, small molecules, synthetic compounds, natural compounds or combinations thereof.

In certain embodiments, the method of preventing or inhibiting a T helper 17 (T_{H}17) cellular response in a subject, comprises administering to the subject a pharmaceutical composition comprising a therapeutically effective amount of an inhibitor of interleukin-17 (IL-17) activity or function. In certain embodiments, the inhibitor of IL-17 inhibits expression of p16, p21, IL-17, type I collagen, S100a4 or combinations thereof. In certain embodiments, the method further comprises administering a senolytic agent, an inhibitor of interleukin-6 (IL-6), an inhibitor of interleukin 1β(IL-1β) or combinations thereof. In certain embodiments, cytokines which reduce T_{H}17 cells can also be administered. The cytokines comprise interleukin-4 (IL-4), interferon-gamma (IFN-γ), interleukin-12 (IL-12), (IL-27) or combinations thereof.

In certain embodiments, a composition comprises a therapeutically effective amount of an IL-17 inhibitory agent, a senolytic agent or a combination thereof. In certain embodiments, the composition further comprises a senomorphic agent. In certain embodiments, the senolytic agent, the senomorphic agent or the IL-17 inhibitory agent comprise: antibodies, antibody fragments, oligonucleotides, polynucleotides, antisense oligonucleotides, enzymes, gene editing agents, nucleases, peptides, polypeptides, small molecules, synthetic compounds, natural compounds or combinations thereof.

In certain embodiments, senolytic agents comprise: dasatinib, quercetin, ABT-263 (navitoclax), ABT-737, piperlongumine (PL), fisetin, HSP90 inhibitors, A1331852, A1155463, ATTAC, BCL-X_{L} inhibitors, or combinations thereof.

In certain embodiments, the senomorphic agent is an inhibitor of the function or expression of suppressor of expression senescent-associated secretory phenotype (SASP) factors, comprising rapamycin, NF-κB and JAK inhibitor, or combinations thereof.

In certain embodiments, the composition further comprises cytokines comprising: interleukin-4 (IL-4), interferon-gamma (IFN-γ), interleukin-12 (IL-12), (IL-27) or combinations thereof.

In certain embodiments, a composition comprises a therapeutically effective amount of an IL-17 inhibitory agent and a senolytic agent. In certain embodiments, the senolytic agent or the IL-17 inhibitory agent comprise: antibodies, antibody fragments, oligonucleotides, polynucleotides, antisense oligonucleotides, enzymes, gene editing agents, nucleases, peptides, polypeptides, small molecules, synthetic compounds, natural compounds or combinations thereof. In certain embodiments, senolytic agents comprise: dasatinib, quercetin, ABT-263 (navitoclax), ABT-737, piperlongumine (PL), fisetin, HSP90 inhibitors, A1331852, A1155463, ATTAC, BCL-XL inhibitors, or combinations thereof. In certain embodiments, the composition further comprises a senomorphic agent, cytokines or a combination thereof In certain embodiments, the cytokines comprise interleukin-4 (IL-4), interferon-gamma (IFN-γ), interleukin-12 (IL-12), (IL-27) or combinations thereof.

Other aspects are described infra.

Definitions

Unless defined otherwise, all technical and scientific terms used herein have the same meaning as commonly understood by one of ordinary skill in the art to which the invention pertains. Although any methods and materials similar or equivalent to those described herein can be used in the practice for testing of the present invention, the preferred materials and methods are described herein. In describing and claiming the present invention, the following terminology will be used. It is also to be understood that the terminology used herein is for the purpose of describing particular embodiments only, and is not intended to be limiting.

The articles “a” and “an” are used herein to refer to one or to more than one (i.e., to at least one) of the grammatical object of the article. By way of example, “an element” means one element or more than one element. Thus, recitation of “a cell”, for example, includes a plurality of the cells of the same type. Furthermore, to the extent that the terms “including”, “includes”, “having”, “has”, “with”, or variants thereof are used in either the detailed description and/or the claims, such terms are intended to be inclusive in a manner similar to the term “comprising.”

“About” as used herein when referring to a measurable value such as an amount, a temporal duration, and the like, is meant to encompass variations of +/−20%, +/−10%, +/−5%, +/−1%, or +/−0.1% from the specified value, as such variations are appropriate to perform the disclosed methods. Alternatively, particularly with respect to biological systems or processes, the term can mean within an order of magnitude within 5-fold, and also within 2-fold, of a value. Where particular values are described in the application and claims, unless otherwise stated the term “about” meaning within an acceptable error range for the particular value should be assumed.

As used herein, the term “agent” or “inhibitor of” or “inhibitory agent” is meant to encompass any molecule, chemical entity, composition, drug, therapeutic agent, chemotherapeutic agent, or biological agent capable of preventing, ameliorating, or treating a dysfunction or other medical condition. The term includes small molecule compounds, antisense oligonucleotides, siRNA reagents, antibodies, antibody fragments bearing epitope recognition sites, such as Fab, Fab′, F(ab′)₂ fragments, Fv fragments, single chain antibodies, antibody mimetics (such as DARPins, affibody molecules, affilins, affitins, anticalins, avimers, fynomers, Kunitz domain peptides and monobodies), peptoids, aptamers; enzymes, gene editing agents, nucleases, peptides organic or inorganic molecules, natural or synthetic compounds and the like. An agent can be assayed in accordance with the methods of the invention at any stage during clinical trials, during pre-trial testing, or following FDA-approval.

By “analog” is meant a molecule that is not identical, but has analogous functional or structural features. For example, a polypeptide analog retains the biological activity of a corresponding naturally-occurring polypeptide, while having certain biochemical modifications that enhance the analog's function relative to a naturally occurring polypeptide. Such biochemical modifications could increase the analog's protease resistance, membrane permeability, or half-life, without altering, for example, ligand binding. An analog may include an unnatural amino acid. “Analogs” in reference to nucleosides includes synthetic nucleosides having modified base moieties and/or modified sugar moieties, e.g., described generally by Scheit, Nucleotide Analogs, John Wiley, New York, 1980; Freier & Altmann, Nucl. Acid. Res., 1997, 25(22), 4429-4443, Toulmé, J. J., Nature Biotechnology 19:17-18 (2001); Manoharan M., Biochemica et Biophysica Acta 1489:117-139(1999); Freier S. M., Nucleic Acid Research, 25:4429-4443 (1997), Uhlman, E., Drug Discovery & Development, 3: 203-213 (2000), Herdewin P., Antisense & Nucleic Acid Drug Dev., 10:297-310 (2000)); 2′-O, 3′-C-linked [3.2.0] bicycloarabinonucleosides (see e.g. N. K Christiensen., et al., J. Am. Chem. Soc., 120: 5458-5463 (1998). Such analogs include synthetic nucleosides designed to enhance binding properties, e.g., duplex or triplex stability, specificity, or the like.

The invention includes antibodies or fragments of such antibodies, so long as they exhibit the desired biological activity. Also included in the invention are chimeric antibodies, such as humanized antibodies. Generally, a humanized antibody has one or more amino acid residues introduced into it from a source that is non-human. Humanization can be performed, for example, using methods described in the art, by substituting at least a portion of a rodent complementarity-determining region for the corresponding regions of a human antibody.

The term “antibody” or “immunoglobulin” is intended to encompass both polyclonal and monoclonal antibodies. The preferred antibody is a monoclonal antibody reactive with the antigen. The term “antibody” is also intended to encompass mixtures of more than one antibody reactive with the antigen (e.g., a cocktail of different types of monoclonal antibodies reactive with the antigen). The term “antibody” is further intended to encompass whole antibodies, biologically functional fragments thereof, single-chain antibodies, and genetically altered antibodies such as chimeric antibodies comprising portions from more than one species, bifunctional antibodies, antibody conjugates, humanized and human antibodies. Biologically functional antibody fragments, which can also be used, are those peptide fragments derived from an antibody that are sufficient for binding to the antigen. “Antibody” as used herein is meant to include the entire antibody as well as any antibody fragments (e.g. F(ab′)₂, Fab′, Fab, Fv) capable of binding the epitope, antigen, or antigenic fragment of interest.

By “antisense oligonucleotides” or “antisense compound” is meant an RNA or DNA molecule that binds to another RNA or DNA (target RNA, DNA). For example, if it is an RNA oligonucleotide it binds to another RNA target by means of RNA-RNA interactions and alters the activity of the target RNA. An antisense oligonucleotide can upregulate or downregulate expression and/or function of a particular polynucleotide. The definition is meant to include any foreign RNA or DNA molecule which is useful from a therapeutic, diagnostic, or other viewpoint. Such molecules include, for example, antisense RNA or DNA molecules, interference RNA (RNAi), micro RNA, decoy RNA molecules, siRNA, enzymatic RNA, short, hairpin RNA (shRNA), therapeutic editing RNA and agonist and antagonist RNA, antisense oligomeric compounds, antisense oligonucleotides, external guide sequence (EGS) oligonucleotides, alternate splicers, primers, probes, and other oligomeric compounds that hybridize to at least a portion of the target nucleic acid. As such, these compounds may be introduced in the form of single-stranded, double-stranded, partially single-stranded, or circular oligomeric compounds.

The term “co-administer” refers to the simultaneous presence of two active agents in the blood of an individual. Active agents that are co-administered can be concurrently or sequentially delivered.

As used herein, the terms “comprising,” “comprise” or “comprised,” and variations thereof, in reference to defined or described elements of an item, composition, apparatus, method, process, system, etc. are meant to be inclusive or open ended, permitting additional elements, thereby indicating that the defined or described item, composition, apparatus, method, process, system, etc. includes those specified elements—or, as appropriate, equivalents thereof—and that other elements can be included and still fall within the scope/definition of the defined item, composition, apparatus, method, process, system, etc.

“Diagnostic” or “diagnosed” means identifying the presence or nature of a pathologic condition. Diagnostic methods differ in their sensitivity and specificity. The “sensitivity” of a diagnostic assay is the percentage of diseased individuals who test positive (percent of “true positives”). Diseased individuals not detected by the assay are “false negatives.” Subjects who are not diseased and who test negative in the assay, are termed “true negatives.” The “specificity” of a diagnostic assay is 1 minus the false positive rate, where the “false positive” rate is defined as the proportion of those without the disease who test positive. While a particular diagnostic method may not provide a definitive diagnosis of a condition, it suffices if the method provides a positive indication that aids in diagnosis.

An “effective amount” as used herein, means an amount which provides a therapeutic or prophylactic benefit.

The term “expression” as used herein is defined as the transcription and/or translation of a particular nucleotide sequence driven by its promoter.

By “fragment” is meant a portion of a polypeptide or nucleic acid molecule. This portion contains, preferably, at least 10%, 20%, 30%, 40%, 50%, 60%, 70%, 80%, or 90% of the entire length of the reference nucleic acid molecule or polypeptide. For example, a fragment may contain 10, 20, 30, 40, 50, 60, 70, 80, 90, or 100, 200, 300, 400, 500, 600, 700, 800, 900, or 1000 nucleotides or amino acids. However, the invention also comprises polypeptides and nucleic acid fragments, so long as they exhibit the desired biological activity of the full length polypeptides and nucleic acid, respectively. A nucleic acid fragment of almost any length is employed. For example, illustrative polynucleotide segments with total lengths of about 10,000, about 5,000, about 3,000, about 2,000, about 1,000, about 500, about 200, about 100, about 50 base pairs in length (including all intermediate lengths) are included in many implementations of this invention. Similarly, a polypeptide fragment of almost any length is employed. For example, illustrative polypeptide segments with total lengths of about 10,000, about 5,000, about 3,000, about 2,000, about 1,000, about 5,000, about 1,000, about 500, about 200, about 100, or about 50 amino acids in length (including all intermediate lengths) are included in many implementations of this invention.

As used herein, the term “immune cells” generally includes white blood cells (leukocytes) which are derived from hematopoietic stem cells (HSC) produced in the bone marrow “Immune cells” includes, e.g., lymphocytes (T cells, B cells, natural killer (NK) cells) and myeloid-derived cells (neutrophil, eosinophil, basophil, monocyte, macrophage, dendritic cells).

The term “immune effector cell,” as used herein, refers to a cell that is involved in an immune response, e.g., in the promotion of an immune effector response. Examples of immune effector cells include T cells, e.g., alpha/beta T cells and gamma/delta T cells, B cells, natural killer (NK) cells, natural killer T (NK-T) cells, mast cells, and myeloid-derived phagocytes. “Immune effector function or immune effector response,” as that term is used herein, refers to function or response, e.g., of an immune effector cell, that enhances or promotes an immune attack of a target cell. For example, an immune effector function or response refers a property of a T or NK cell that promotes killing or the inhibition of growth or proliferation, of a target cell. In the case of a T cell, primary stimulation and co-stimulation are examples of immune effector function or response.

By “marker” is meant any protein or polynucleotide having an alteration in expression level or activity that is associated with a disease or disorder.

The terms “preventing” and “prevention” refer to the administration of an agent or composition to a clinically asymptomatic individual who is susceptible or predisposed to a particular adverse condition, disorder, or disease, and thus relates to the prevention of the occurrence of symptoms and/or their underlying cause.

As used in this specification and the appended claims, the term “or” is generally employed in its sense including “and/or” unless the content clearly dictates otherwise.

“Parenteral” administration of an immunogenic composition includes, e.g., subcutaneous (s.c.), intravenous (i.v.), intramuscular (i.m.), or intrasternal injection, or infusion techniques.

The terms “patient” or “individual” or “subject” are used interchangeably herein, and refers to a mammalian subject to be treated, with human patients being preferred. In some cases, the methods of the invention find use in experimental animals, in veterinary application, and in the development of animal models for disease, including, but not limited to, rodents including mice, rats, and hamsters, and primates.

As used herein, a “pharmaceutically acceptable” component/carrier etc. is one that is suitable for use with humans and/or animals without undue adverse side effects (such as toxicity, irritation, and allergic response) commensurate with a reasonable benefit/risk ratio.

“Cellular senescence” is a cell fate that involves essentially irreversible replicative arrest, apoptosis resistance, and frequently increased protein synthesis, metabolic shifts with increased glycolysis, decreased fatty acid oxidation, increased reactive oxygen species generation, and acquisition of a senescence-associated secretory phenotype (SASP). The SASP entails secretion of cytokines, bradykines, prostenoids, miRNA's, damage-associated molecular pattern proteins (DAMPs), and other pro-inflammatory mediators, chemokines that attract immune cells, factors that cause stem cell dysfunction such as activin A, hemostatic factors such as PAI-1, pressors, and extracellular matrix-damaging molecules, including proteases. Senescence can occur in response to potentially oncogenic mutations, activated oncogenes, metabolic insults, and damage/danger signals (Kirkland J. L., et al. EBioMedicine, Volume 21, July 2017, Pages 21-28). Senescent Cell Anti-apoptotic Pathways (SCAPs) shield senescent cells from their own pro-apoptotic SASP.

A “senotherapeutic agent” specifically kills senescent cells (“senolytic agents”) or suppress the senescence-associated secretory phenotype (SASP) that drives sterile inflammation (“senomorphic agents”) associated with aging to extend healthspan and potentially lifespan.

By “siRNA” is meant a double stranded RNA. Optimally, an siRNA is 18, 19, 20, 21, 22, 23 or 24 nucleotides in length and has a 2 base overhang at its 3′ end. These dsRNAs can be introduced to an individual cell or to a whole animal; for example, they may be introduced systemically via the bloodstream. Such siRNAs are used to downregulate mRNA levels or promoter activity.

As defined herein, a “therapeutically effective” amount of a compound or agent (i.e., an effective dosage) means an amount sufficient to produce a therapeutically (e.g., clinically) desirable result. The compositions can be administered from one or more times per day to one or more times per week; including once every other day. The skilled artisan will appreciate that certain factors can influence the dosage and timing required to effectively treat a subject, including but not limited to the severity of the disease or disorder, previous treatments, the general health and/or age of the subject, and other diseases present. Moreover, treatment of a subject with a therapeutically effective amount of the compounds of the invention can include a single treatment or a series of treatments.

As used herein, “treating” or “treatment” and grammatical variants thereof refer to an approach for obtaining beneficial or desired clinical results. The term may refer to slowing the onset or rate of development of a condition, disorder or disease, reducing or alleviating symptoms associated with it, generating a complete or partial regression of the condition, or some combination of any of the above. For the purposes of this invention, beneficial or desired clinical results include, but are not limited to, reduction or alleviation of symptoms, diminishment of extent of disease, stabilization (i.e., not worsening) of state of disease, delay or slowing of disease progression, amelioration or palliation of the disease state, and remission (whether partial or total), whether detectable or undetectable. “Treatment” can also mean prolonging survival relative to expected survival time if not receiving treatment. A subject (e.g., a human) in need of treatment may thus be a subject already afflicted with the disease or disorder in question. The term “treatment” includes inhibition or reduction of an increase in severity of a pathological state or symptoms relative to the absence of treatment, and is not necessarily meant to imply complete cessation of the relevant disease, disorder or condition.

The practice of the present invention employs, unless otherwise indicated, conventional techniques of chemistry, molecular biology, microbiology, recombinant DNA, genetics, immunology, cell biology, cell culture and transgenic biology, which are within the skill of the art. See, e.g., Maniatis et al., 1982, Molecular Cloning (Cold Spring Harbor Laboratory Press, Cold Spring Harbor, N.Y.); Sambrook et al., 1989, Molecular Cloning, 2nd Ed. (Cold Spring Harbor Laboratory Press, Cold Spring Harbor, N.Y.); Sambrook and Russell, 2001, Molecular Cloning, 3rd Ed. (Cold Spring Harbor Laboratory Press, Cold Spring Harbor, N.Y.); Ausubel et al., 1992), Current Protocols in Molecular Biology (John Wiley & Sons, including periodic updates); Glover, 1985, DNA Cloning (IRL Press, Oxford); Anand, 1992; Guthrie and Fink, 1991; Harlow and Lane, 1988, Antibodies, (Cold Spring Harbor Laboratory Press, Cold Spring Harbor, N.Y.); Jakoby and Pastan, 1979; Nucleic Acid Hybridization (B. D. Hames & S. J. Higgins eds. 1984); Transcription And Translation (B. D. Hames & S. J. Higgins eds. 1984); Culture Of Animal Cells (R. I. Freshney, Alan R. Liss, Inc., 1987); Immobilized Cells And Enzymes (IRL Press, 1986); B. Perbal, A Practical Guide To Molecular Cloning (1984); the treatise, Methods In Enzymology (Academic Press, Inc., N.Y.); Gene Transfer Vectors For Mammalian Cells (J. H. Miller and M. P. Calos eds., 1987, Cold Spring Harbor Laboratory); Methods In Enzymology, Vols. 154 and 155 (Wu et al. eds.), Immunochemical Methods In Cell And Molecular Biology (Mayer and Walker, eds., Academic Press, London, 1987); Handbook Of Experimental Immunology, Volumes I-IV (D. M. Weir and C. C. Blackwell, eds., 1986); Riott, Essential Immunology, 6th Edition, Blackwell Scientific Publications, Oxford, 1988; Hogan et al., Manipulating the Mouse Embryo, (Cold Spring Harbor Laboratory Press, Cold Spring Harbor, N.Y., 1986); Westerfield, M., The zebrafish book. A guide for the laboratory use of zebrafish (Danio rerio), (4th Ed., Univ. of Oregon Press, Eugene, 2000).

Genes: All genes, gene names, and gene products disclosed herein are intended to correspond to homologs from any species for which the compositions and methods disclosed herein are applicable. It is understood that when a gene or gene product from a particular species is disclosed, this disclosure is intended to be exemplary only, and is not to be interpreted as a limitation unless the context in which it appears clearly indicates. Thus, for example, for the genes or gene products disclosed herein, are intended to encompass homologous and/or orthologous genes and gene products from other species.

Ranges: throughout this disclosure, various aspects of the invention can be presented in a range format. It should be understood that the description in range format is merely for convenience and brevity and should not be construed as an inflexible limitation on the scope of the invention. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 2.7, 3, 4, 5, 5.3, and 6. This applies regardless of the breadth of the range.

Any compositions or methods provided herein can be combined with one or more of any of the other compositions and methods provided herein.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

FIGS. 1A-1C are a series of plots, immunostains and graphs demonstrating that IL-17 is secreted by gamma-delta and CD4⁺ T cells in tissue surrounding human breast implants and correlates with expression of fibrosis. Tissue samples surrounding silastic tissue expanders and implants were evaluated by flow cytometry and gene expression analysis. FIG. 1A: Flow cytometry revealed T helper cells (CD45⁺Thy 1.2⁺CD3⁺CD4⁺) T cells and γδ⁺ T cells (CD45⁺Thy 1.2⁺CD3⁺γδ⁺) from implant-associated tissue produced IL-17 at significantly greater levels compared to interferon gamma (IFNγ), and interleukin 4 (IL-4). The percentage of IL-17a⁺ cells in both populations was higher compared to IFNγ or IL-4 secreting cells. Tissues from each patient are designated with different shapes. FIG. 1B: Immunofluorescence staining of pSTAT3 (green) and IL-17 (red) in the fibrous capsule. Masson's trichrome and hematoxylin & eosin staining showed the morphology and infiltration of immune cells. FIG. 1C: Correlation of qRT-PCR gene expression between IL-17a mRNA and fibrosis-associated genes, including Col1a1, Col3a1, TGFβ. Data are means±SD, n=3 (FIG. 1A), n=8 (FIG. 1C) ANOVA [(FIG. 1A) and (FIG. 1C)]: ****P<0.0001, ***P<0.001, **P<0.01, *P<0.05.

FIGS. 2A-2G are a series of graphs and plots demonstrating that synthetic materials induce an IL-17 response in muscle tissue. FIG. 2A: C56BL/6 mice received quadricep muscle injuries and were subsequently implanted with synthetic biomaterial particles or saline. Numbers of IFNγ, IL4, and IL17A secreting T cells were quantified at different time points. FIG. 2B: Kinetics of IL17A expression by different cell types, including innate lymphoid cells (ILCs), γδ+ T cells, and CD4+ T helper cells over time. FIG. 2C: Representative flow cytometry plots of IL17A production by CD4+ T cells at 3 and 6-weeks post-surgery. FIG. 2D: IL17A and IL17F cytokines were quantified in ILCs and TH17 cells by flow cytometry in muscle implanted with PCL and PE at 3-weeks post-injury. FIG. 2E: qRT-PCR gene expression of Il117a and other inflammatory genes such as Il1β, Il23, and Tnfα in tissue surrounding PCL, PEG, or silicone implants 6-weeks after implantation. FIG. 2F: Experimental overview of the bone marrow chimera. CD45.1 and CD45.2 were evaluated. FIG. 2G: Splenocytes from OT-II transgenic mice cultured with different materials (PCL or PE) and OVA antigen. Production of IL17A was quantified via intracellular staining. Data are displayed as relative quantification (RQ) to healthy tissue controls. Data are means±SD, n=4-8 [(A), (B)], n=4 [(D), (E), and (F)], n=3 (G). ANOVA [(A), (B), (D), (E), (F) and (G)]: ****P<0.0001, ***P<0.001, **P<0.01, *P<0.05.

FIGS. 3A-3D are a series of histological stains, immunofluorescence and graphs demonstrating that IL-17 inhibition reduces the fibrotic response to synthetic particle. FIG. 3A: PCL was implanted in knockout mice lacking IL17A expression and IL17 signaling through the IL17RA receptor. Histological staining of implants 12-weeks for collagen (Masson's trichrome) and immunofluorescence for the macrophage marker F4/80 and fibrosis-associated αSMA protein were evaluated. FIG. 3B: qRT-PCR gene expressions of fibrotic markers including TGFβ, S100a4, and collagen III were analyzed in WT, IL17A−/−, and IL17RA−/− mice at 6-weeks post-surgery. FIG. 3C: Co-administration of IL17A and IL17F neutralizing antibody (100 μg/mL each) or isotype control (mouse IgG1) was given intraperitoneally to mice with PCL implanted for 4 weeks. To evaluate the degree of fibrosis, tissues was harvested at week 6 for histological assessment. Immunofluorescence staining showed αSMA markers in mice with antibody treatment compared to isotype control. Picrosirius red stain showed the spectrum of color (green to red) relative to the degree of collagen density (thinnest to thickest respectively) and green to red illuminant was quantified by CIELAB. Thickness of the fibrous capsule in WT and IL17 deficient mice was also measured. FIG. 3D: qRT-PCR gene analysis of Il17a, Il6, Tgfβ, and type I collagen in tissues treated with neutralizing antibody compared to isotype control at 6-weeks post-injury. Data are displayed as relative quantification (RQ) and saline control. Data are means±SD, n=9 (WT mice), n=4 (IL17 knockout mice), ANOVA [(B), (C) and (D)]: ****P<0.0001, ***P<0.001, **P<0.01, *P<0.05.

FIGS. 4A-4G are a series of immunofluorescent stains and graphs demonstrating that synthetic scaffold in wild type mice induced a potent senescence associated marker, p16^{INK4a}, whereas IL-17a^{-/-} and IL-17ra^{-/-} mice did not. FIG. 4A: qRT-PCR gene expression of p16INK4a progression normalized to healthy tissue control over time. FIG. 4B: Immunofluorescence staining of p16INK4a (red) in wild type mice, IL17A−/−, and IL17RA−/− mice. FIG. 4C: qRT-PCR analyses of p16INK4a comparing WT mice to IL17A−/− and IL17RA−/− mice with synthetic implants 12-weeks post-injury. FIG. 4D: Gene expression of p16INK4a comparing different synthetic materials 6-weeks post-injury. FIG. 4E: indicated the 95% confidence intervals (CI) of the fitted line. FIG. 4F: Navitoclax (ABT-263, or Navi) was administrated to eliminate senescent cells. Images of αSMA expression in vehicle (5% DMSO, 3% Tween 80 in PBS) and Navi treated mice, followed by quantification of G-R illuminant by CIELAB using PSR. FIG. 4G: Mice received Navi treatment alone or in combination with IL17A/F neutralizing antibody and were harvested at 6-weeks post-injury to evaluate gene expression of p16INK4a, Il17a, Il6, Il4, Tgfβ, S100a4, collagen type I and III. Data are means±SD, n=4 [(A), (C), (D), (E), (G)], n=8 (F). ANOVA [(A) (C) (D) (E) and (G)], linear regression (F): ****P<0.0001, ***P<0.001, **P<0.01, *P<0.05.

FIGS. 5A and 5B are plots showing the flow cytometry gating strategies for myeloid (FIG. 5A) and lymphoid (FIG. 5B) populations.

FIGS. 6A-6F show that CD3+ T cells are highly upregulated in human fibrotic capsule tissue. FIG. 6A is a schematic illustration of human fibrotic capsule extraction via mastectomy procedure and gross images of the implants. FIGS. 6B, 6C Representative flow cytometry plot and quantification of myeloid derived cells, including monocytes (CD3−CD11c−CD14+CD16−), granulocytes (CD3−CD11c−CD14+CD16+CD15+), eosinophils (CD3−CD11c−CD14+CD16+CD15−), dendritic cells (CD3-CD11c+), and lymphoid derived T cells (CD3+CD11c−). FIG. 6D: Quantification of the percentage of CD4+ T cells and γδ+ T cells in 5 patients. FIG. 6E: Immunofluorescence imaging showing pSTAT3 and IL17 in human fibrous capsule, followed by single staining controls and primary delete. FIG. 6F: Correlation of qRT-PCR gene expression between Il17a mRNA and fibrosis-associated genes, including S100a4, Ifnγ, Il4, and Il6. Data are means±SD, n=3 (C), n=8 (D), n=5 (F) ANOVA [(C) (D) and (F)]: ****P<0.0001,***P<0.001, **P<0.01, *P<0.05.

FIGS. 7A-7E. FIG. 7A: A schematic illustration of volumetric muscle loss (VML) and subcutaneous (SQ) implants in the murine model. FIG. 7B: Flow cytometry analysis of the total number of ILCs, γδ+ T cells, and CD3+ T cells at 1, 3, and 6- weeks post-surgery. FIG. 7C: Kinetics of IFNγ and IL4 expression by ILCs, γδ+ T cells, and CD4+ T helper cells at various post-injury time points. FIG. 7D: Quantification of IL17A expression across different cell types 3-weeks post-surgery in an IL17A-GFP reporter mice. FIG. 7E: Comparison of CD4+ and CD8+ T cells at 3-weeks post-injury with PCL and PE implant. Data are means±SD, n=8 (B, C), n=3 (D), n=6 (E) ANOVA [(B), (C), (D) and (E)]: ****P<0.0001, ***P<0.001,**P<0.01, *P<0.05.

FIGS. 8A-8D show the xpression prolife of immune cells from synthetic implant is proinflammatory. FIG. 8A: CD3+ T cells were sorted for gene expression analysis using the NanoString platform. Volcano plot of genes differentially regulated in PCL-derived T cells compared to saline (no implant) control at day 7 post-surgery. Type 17-associated gene expression differences are highlighted. FIG. 8B: qRT-PCR gene expression normalized to healthy muscle control showing kinetics of Il4, Il10, Il17a, Il23, Tnfα, and Il1β transcripts. FIG. 8C: Gene expression analysis comparing levels of different cytokines between the VML model and subcutaneous (SQ) implant, and intramuscular injection (IM) model at 1-week post-injury. FIG. 8D: Total number of CD4 and CD8 infiltration, TH1, and TH2, Tc1, Tc17 from CD45.1 (WT donor) and CD45.2 (OTII-Rag−/− donor) bone marrow chimera mice 1 week after VML. Data are means±SEM (B), means±SD, n=4 (B), n=6 (C), n=4 (D) ANOVA [(B), (C) and (D)]: ****P<0.0001, ***P<0.001, **P<0.01, *P<0.05.

FIGS. 9A-9D illustrate that the Type 17 response induced by synthetic material is antigen dependent. FIG. 9A: IgG1, IgM, and IgA antibody titers were detected using an enzyme-linked immunosorbent assay (ELISA) system on serum collected 1, 3, and 6-weeks post-surgery and implantation. FIG. 9B: Gene expressions of Il17a on SubQ implant in mice previously primed with PCL implant in VML. In addition, neutralizing antibody IL6 (100 μg/mL each mouse per day for 5 consecutive days) or isotype control (rat IgG1) was administered intraperitoneally at 2-weeks post-VML. FIG. 9C: Spleenocytes were isolated from mice received initial challenge with PCL implant or saline control. Cells were labeled with CellTrace Violet proliferation dye. In culture, PCL were re- introduced to investigate the memory response. Proliferation were evaluated via flow cytometry at 24, 48, 72, and 96 hours. FIG. 9D: qRT-PCR gene expression normalized to healthy muscle controls showing kinetics of Tgfβ, S100a4, type I and III collagen progression in PCL implant mice compared to saline control overtime. Data are means±SD, means±SD, n=4, ANOVA [(A), (B), (C) and (D)]: ****P<0.0001, ***P<0.001, **P<0.01, *P<0.05.

FIGS. 10A-10E shows that the Type 17 response regulates the recruitment of myeloid cells. FIG. 10A: Gene expression analysis of F4/80⁺ cells sorted from PCL implanted WT mice 6-weeks post-surgery. FIG. 10B: Flow cytometry representative plots comparing different populations of myeloid cells in WT, IL17A^{-/-}, and IL17RA^{-/-} mice. FIG. 10C: Quantification of granulocytes (CD11b⁺Ly6intLy6g^{high}), MHCII^{high} macrophages (CD11b⁺Ly6c^{low}Ly6g^{low}MHCII^{high}F4/80⁺), MHCII^{low} macrophages (CD11b⁺Ly6c^{low}Ly6g^{low}MHCII^{low}F4/80⁺), and monocytes (CD11b⁺Ly6c^{high}Ly6g^{low}) in WT, IL17A^{-/-}, and IL17RA^{-/-} mice after 3 weeks with PCL implants. FIG. 10D: Immunofluorescence imaging of CD11b and Ly6g around PCL implants in WT and IL17 signaling deficient mice 12-weeks post-surgery. FIG. 10E: Immunofluorescence imaging of F4/80 and p16^{INK4a} in WT mice 12-weeks post-surgery. Data are means±SD, n=4, ANOVA [(A), (C)]: ****P<0.0001, ***P<0.001, **P<0.01, *P<0.05.

FIGS. 11A-11D illustrates that IL17 induces senescent fibroblasts. FIG. 11A: Immunofluorescence staining of p16^{INK4a} (red) in WT, IL17A^{-/-}, and IL17RA^{-/-}, OTII-Rag^{-/-} mice at 6-weeks post-injury, followed by primary antibody delete control. FIG. 11B: Gene expression analysis of fibroblasts sorted from PCL implanted WT mice 6-weeks post-surgery. FIG. 11C: Gene expressions of Il17a in mice that received injection of normal fibroblast or senescent fibroblasts compared to no injection control. FIG. 11D: Gene expressions of Il17a at 1-week post-VML in IL6^{-/-} mice that received injection of senescent fibroblasts compared to normal fibroblasts. Data are means±SD, n=4 (B, D), n=3 (C), ANOVA [(B), (C), and (D)]: ****P<0.0001, ***P<0.001, **P<0.01, *P<0.05.

FIGS. 12A-12C show that systemic immune homeostasis is influenced by application of synthetic implant. FIG. 12A: Total number of immune cells infiltration and TH17 from CD45.1 (WT donor) and CD45.2 (OTII-Rag−/− donor) bone marrow chimera mice 1 week after VML. Percent of CD4+ T cells, TH1, TH2, and TH17 cells from CD45.1 and CD45.2 in draining lymph nodes were evaluated. FIG. 12B: In vitro differentiation of TH17 cells of OTII-Rag−/− compared to WT naïve CD4+ cells. FIG. 12C: Proliferation assay of T cells co-cultured with spleenocytes from OT-II transgenic mice cultured with different materials (PCL or PE) and OVA antigen. Representative plots for proliferation assay were shown. Data are means±SD, n=4 (A), n=3 (C), ANOVA [(A) and (C)]: ****P<0.0001, ***P<0.001, **P<0.01, *P<0.05.

FIGS. 13A-13C show that IL17 deficient mice reduces the fibrotic response to synthetic materials. FIG. 13A: Gene expression analysis of fibrotic markers in the whole tissue including Tgfβ, S100a4, and type III collagen in WT, IL17A−/−, and IL17RA−/− mice at 12-weeks post-surgery. FIG. 13B: Treadmill exhaustion assay 3, 6, and 12-weeks post-implantation of WT, IL17A−/−, and IL17RA−/− mice. FIG. 13C: qRT-PCR analysis of inflammasome related genes including Il1β and Nlrp3 was performed in WT, IL17A−/−, and IL17RA−/− mice at 12-weeks post-surgery. FIG. 13D: Gene expressions of Smad2, Smad3, and Smad4 in WT, IL17A−/−, and IL17RA−/− mice at 6-weeks post-surgery were shown.

Data are means±SD (B), means±SD, n=4 [(A), (C), (D)], n=5 (B), ANOVA [(A), (B), (C) and (D)]: ****P<0.0001, ***P<0.001, **P<0.01, *P<0.05.

### DETAILED DESCRIPTION

T cells are a key component of the adaptive immune system that is increasingly recognized for their role in wound healing and tissue repair. CD4+ helper T cells regulate bone, liver, and muscle repair processes (11-13). The TH2 T effector cells responding to pro-regenerative biological scaffolds secrete interleukin 4 (IL4) and direct the function of macrophages to promote muscle repair (13). The presence of T cells has been recognized in the FBR in animal models and surrounding clinical implants but their nature, activation status and role in the response is still largely unknown (10, 14). Adaptive responses that depend on T cells, which conventionally recognize MHC-presented peptide antigens, have not been seriously considered in the response to synthetic materials despite their increasing association with fibrotic disease (15-17). Beyond T cells, the influence of other immune cell types such as gamma-delta (γδ) T cells and innate lymphoid cells (ILCs) in regulation of the biomaterial response, tissue damage, and fibrosis remains unexplored.

### Compositions

The invention is based in part on the discovery that ILCs, γδ⁺ and CD4⁺ T cells are the primary sources of IL-17 that promote a fibrotic response to biomaterials. The interplay between IL-17 and cellular senescence was established herein, as a mechanism linking the chronic immune response to synthetic implants to excessive fibrosis, a novel concept that introduces immune-stromal interactions as therapeutic target.

Accordingly, in certain embodiments compositions for the prevention of foreign body responses (FBR) and/or the treatment of such immune responses comprise a therapeutically effective amount of an interleukin-17 (IL-17) inhibitor.

T helper 17 (T_{H}17 cells). T helper (T_{H}) cells that secrete interleukin-17 (IL-17), called T_{H}17 cells, are a subpopulation of CD4⁺ T cells that are involved in the disease progression of many autoimmune and inflammatory disorders due to their secretion of the IL-17 family cytokines IL-17A and IL-17F as well as IL-22 and granulocyte-macrophage colony-stimulating factor (GM-CSF). The key to T_{H}17 differentiation in the mouse is the combination of transforming growth factor-β (TGF-β) and IL-6. In addition, tumor necrosis factor-a (TNF-α) and IL-1β can further enhance mouse T_{H}17 differentiation, but only in the presence of TGF-β and IL-6. T_{H}17 cells appear to be resistant to the suppressive effects of Tregs (Annunziato F, et al. Phenotypic and functional features of human Th17 cells. J Exp Med. 2007; 204:1849-1861; Evans H G, et al. Optimal induction of T helper 17 cells in humans requires T cell receptor ligation in the context of Toll-like receptor-activated monocytes. Proc Natl Acad Sci USA. 2007; 104:17034-17039).

When T cells differentiate, they begin to express specific cytokines, such as IFN-γ in T_{H}1 and IL-4 in T_{H}2, which act in an autocrine feedback loop to further promote differentiation, thus giving activated T cells self-sufficiency to move out of the lymphoid tissue and traffic to the site of inflammation while continuing to develop. Similarly, mouse T_{H}17 cells specifically express IL-21 soon after activation, and autocrine IL-21 plays an important role in RORγt and IL-17 expression. IL-21 can also partially replace IL-6 during T_{H}17 differentiation, giving established T_{H}17 cells the ability to promote further T_{H}17 development in neighboring cells. IL-23 in combination with TGF-β can also induce RORγt and IL-17 expression, but only after IL-6 or IL-21 induces IL-23 receptor expression (Wei L, et al. J Biol Chem. 2007; 282:34605-34610; Fantini M C, et al. Eur Jlmmunol. 2007; 37:3155-3163 ; Zhou L, et al. Nat. Immunol. 2007; Korn T, et al. Nature. 2007; 448:484-487; Nurieva R, et al. Nature. 2007; 448:480-483). Thus IL-6, IL-21, and IL-23 act sequentially: first IL-6 upregulates IL-21, then both IL-6 and IL-21 upregulate IL-23 receptor, and finally IL-23 appears to upregulate effector function and pathogenicity in T_{H}17 cells through an unknown mechanism.

Cell induction occurs in three transcriptional phases (Yosef N, et al., Nature. 2013 Apr. 25; 496(7446):461-8). In the first phase, the classic T_{H}17 transcription factor genes Stat3, Irf4, and Batf; the cytokines Il21 and Lif, and cytokine receptors Il2ra and Il23r are induced. In the second phase, the Rorc gene is induced to encode the major regulatory nuclear receptor of the T_{H}17 subtype, RAR-related orphan receptor gamma t (ROR-γt). In the third phase, the phenotypic cytokines of T_{H}17 cells are induced while the cytokines of other subclasses of T cells are suppressed at the transcriptional level (Miossec P, Kolls J K. Nat Rev Drug Discov. 2012 October; 11(10):763-76; Weaver CT, et al., Annu Rev Pathol. 2013 Jan. 24; 8:477-512; Yosef N. et al. Id).

Gamma delta T (γδ T) cells. These T cells are an important subset of “unconventional” T lymphocytes as they have the ability to recognize a broad range of antigens without the presence of major histocompatibility complex (MHC) molecules. They can attack target cells directly through their cytotoxic activity or indirectly through the activation of other immune cells.γδ T-cell functional responses are induced upon the recognition of stress antigens, which promotes cytokine production and regulates pathogen clearance, inflammation, and tissue homeostasis in response to stress (Bonneville M, et al., Nat Rev Immunol. 2010 July; 10(7):467-78).

In humans, there are two major subsets of γδ T cells identified by their Vδ chain. Vδ1 T cells are predominant in the thymus and peripheral tissues and recognize various stress-related antigens mostly uncharacterized. Vδ2 T cells constitute the majority of blood γδ T cells (Vantourout P, Hayday A. Nat Rev Immunol. 2013 February; 13(2):88-100). Both human γδ T-cell subsets exhibit a cytotoxic potential that is induced through the expression of cell surface receptors [i.e., γδ TCR (T-cell receptor) and NKG2D (natural killer group 2D)] and is preponderantly mediated by the release of soluble mediators (i.e., perforin and granzymes) (Wrobel P, et al. Scand J Immunol. 2007 August-September; 66(2-3):320-8; Todaro M, et al. J Immunol. 2009 Jun. 1; 182(11):7287-96). They can produce granulysin, which is a potent anti-microbial protein (Spada F M, et al., J Exp Med. 2000 Mar. 20; 191(6):937-48Costa G, et al., Blood. 2011 Dec. 22; 118(26):6952-62), and express ligands such as CD95L and Tumor necrosis factor-related apoptosis-inducing ligand, which engage several death receptors on target cells. In addition, they can kill their targets indirectly through antibody-dependent cellular cytotoxicity (ADCC) in a CD16-dependent mechanism (Couzi L, et al., Blood. 2012 Feb. 9; 119(6):1418-27). Other molecules such as DNAM-1 (DNAX accessory molecule-1), leukocyte function-associated antigen-1, and the co-stimulatory receptor CD27 are also involved in γδ T-cell activation and cytotoxicity (Silva-Santos B, et al., Nat Rev Immunol. 2015 November; 15(11):683-91).

Importantly, cord blood naïve γδ T cells can differentiate into the IL-17⁺IFN-γ⁻ Vγ9Vδ2 T cells with a cytotoxic potential in the presence of IL-23 and a TCR signaling. In contrast, thymic naïve γδ T cells secrete IFN-γ in the presence of IL-2 or IL-15, through the de novo expression of T-bet and eomesodermin, and the release of cytotoxic molecules against leukemia cells. Other studies reported IL-17⁺ γδ T-lymphocyte differentiation in the presence of IL-7 or other activation stimuli and high inflammatory conditions (Lawand, Myriam et al. Frontiers in Immunology vol. 8 761. 30 Jun. 2017, doi:10.3389/fimmu.2017.00761).

Innate lymphoid cells (ILCs). These cells are a growing family of immune cells that mirror the phenotypes and functions of T cells. Natural killer (NK) cells can be considered the innate counterparts of cytotoxic CD8⁺ T cells, whereas ILC1s, ILC2s, and ILC3s may represent the innate counterparts of CD4⁺ T helper 1 (T_{H}1), T_{H}2, and T_{H}17 cells. However, in contrast to T cells, ILCs do not express antigen receptors or undergo clonal selection and expansion when stimulated. Instead, ILCs react promptly to signals from infected or injured tissues and produce an array of cytokines, that direct the developing immune response into one that is adapted to the original insult (Vivier, Eric et al. “The evolution of innate lymphoid cells” Nature immunology vol. 17,7 (2016): 790-4).

ILCs are categorized into 3 groups based on their distinct patterns of cytokine production and the requirement of particular transcription factors for their development and function. Group 1 ILCs (ILC1s) produce interferon γ and depend on Tbet, group 2 ILCs (ILC2s) produce type 2 cytokines like interleukin-5 (IL-5) and IL-13 and require GATA3, and group 3 ILCs (ILC3s) include lymphoid tissue inducer cells, produce IL-17 and/or IL-22, and are dependent on RORγt (Mette D. Hazenberg and Hergen Spits. Blood 2014 124:700-709).

Foreign Body Response/Reaction: Host reactions following implantation of biomaterials include injury, blood-material interactions, provisional matrix formation, acute inflammation, chronic inflammation, granulation tissue development, foreign body reaction, and fibrosis/fibrous capsule development (Anderson J M, Rodriguez A, Chang D T. Foreign body reaction to biomaterials. Semin Immunol. 2007; 20(2):86-100). In the very early process of implantation, blood/material interactions occur with protein adsorption to the biomaterial surface and development of a blood-based transient provisional matrix that forms on and around the biomaterial. The provisional matrix is the initial thrombus/blood clot at the tissue/material interface. The injury to vascularized connective tissue not only initiates the inflammatory responses (innate immunity), it also leads to thrombus formation involving activation of the extrinsic and intrinsic coagulation systems, the complement system, the fibrinolytic system, the kinin-generating system, and platelets. These protein cascades may be intimately involved in the dynamic phenomenon of protein adsorption and desorption that is known as the Vroman Effect (Horbett T. The role of adsorbed proteins in tissue response to biomaterials. In: Ratner B, et al., editors. Biomaterials Science: An Introduction to Biomaterials in Medicine. San Diego, Calif.: Elsevier Academic Press; 2004. pp. 237-46). From a wound healing perspective, blood protein deposition on a biomaterial surface is described as provisional matrix formation. The provisional matrix furnishes structural, biochemical, and cellular components to the processes of wound healing and foreign body reaction. The presence of mitogens, chemoattractants, cytokines, growth factors, and other bioactive agents within the provisional matrix provides for a rich milieu of activating and inhibiting substances capable of modulating macrophage activity, along with the proliferation and activation of other cell populations in the inflammatory and wound healing responses. The provisional matrix may be viewed as a naturally derived, biodegradable sustained release system in which bioactive agents are released to control subsequent phases of wound healing.

Following the initial blood/material interactions and provisional matrix formation, acute and chronic inflammation occurs in a sequential fashion. The extent or degree of these responses is controlled by the extent of injury in the implantation procedure, the tissue or organ into which the device is implanted, and the extent of provisional matrix formation. Neutrophils (polymorphonuclear leukocytes, PMNs) characterize the acute inflammatory response. Mast cell degranulation with histamine release and fibrinogen adsorption is known to mediate acute inflammatory responses to implanted biomaterials. Interleukin-4 (IL-4) and interleukin-13 (IL-13) also are released from mast cells in a degranulation process and can play significant roles in determining the extent and degree of the subsequent development of the foreign body reaction (Keegan A D. IL-4. In: Oppenheim J J, Feldman M, editors. Cytokine Reference. San Diego, Calif.: Academic Press; 2001; McKenzie A N J, Matthews D J. IL-13. In: Oppenheim J J, Feldman M, editors. Cytokine Reference. San Diego, Calif.: Academic Press; 2001). Biomaterial mediated inflammatory responses may be modulated by histamine-mediated phagocyte recruitment and phagocyte adhesion to implant surfaces facilitated by adsorbed host fibrinogen. Both H1 and H2 histamine receptor antagonists greatly reduce the recruitment of monocytes/macrophages and neutrophils on implant surfaces. Protein adsorption and monocyte/macrophage adhesion are significant topics in the foreign body reaction and are discussed later in this review. The acute inflammatory response with biomaterials usually resolves quickly, usually less than one week, depending on the extent of injury at the implant site.

Following acute inflammation, chronic inflammation is identified by the presence of mononuclear cells, i.e. monocytes and lymphocytes, at the implant site. Chronic inflammation is less uniform histologically than acute inflammation and this term has been used diagnostically to identify a wide range of cellular responses. The presence of mononuclear cells, including lymphocytes and plasma cells, is considered chronic inflammation. This chronic inflammatory response to biomaterials is usually of short duration and is confined to the implant site. Chronic inflammation also has been used to describe the foreign body reaction where monocytes, macrophages, and foreign body giant cells are present at the biomaterial interface. With biocompatible materials, early resolution of the acute and chronic inflammatory responses occurs with the chronic inflammatory response composed of mononuclear cells usually lasting no longer than two weeks. The persistence of the acute and/or inflammatory responses beyond a three week period usually indicates an infection. Following resolution of the acute and chronic inflammatory responses, granulation tissue identified by the presence of macrophages, the infiltration of fibroblasts, and neovascularization in the new healing tissue is identified. Granulation tissue is the precursor to fibrous capsule formation and granulation tissue is separated from the implant or biomaterial by the cellular components of the foreign body reaction; a one- to two-cell layer of monocytes, macrophages, and foreign body giant cells.

Cellular Senescence: Cellular senescence is one phenomenon by which normal cells cease to divide. Mechanistically, replicative senescence is triggered by a DNA damage response which results from the shortening of telomeres during each cellular division process. Cells can also be induced to senesce independent of the number of cellular divisions via DNA damage in response to elevated reactive oxygen species (ROS), activation of oncogenes and cell-cell fusion. The number of senescent cells in tissues rises substantially during normal aging (Childs BG, et al. (2015). Nature Medicine. 21 (12): 1424-1435). Senescent cells accumulate in numerous tissues with aging and at sites of pathogenesis of multiple chronic diseases (Kirkland J L, Tchkonia T. Exp Gerontol. 2015 August; 68:19-25; Zhu Y, et al., Curr Opin Clin Nutr Metab Care. 2014 July; 17(4):324-8). Small numbers of senescent cells can cause extensive local and systemic dysfunction due to their pro-inflammatory senescence-associated secretory phenotype (SASP) (Coppé J P, et al., PLoS Biol. 2008 Dec.2; 6(12):2853-68).

Although senescent cells can no longer replicate, they remain metabolically active and commonly adopt an immunogenic phenotype consisting of a pro-inflammatory secretome, the up-regulation of immune ligands, a pro-survival response, promiscuous gene expression (pGE) and stain positive for senescence-associated β-galactosidase activity. Senescence-associated beta-galactosidase, along with p16^{Ink4A}, is regarded to be a biomarker of cellular senescence. This results in false positives for maturing tissue macrophages and senescence-associated beta-galactosidase as well as for T-cells p16^{Ink4A} (Campisi, Judith (February 2013). “Aging, Cellular Senescence, and Cancer”. Annual Review of Physiology. 75: 685-705). A Senescence Associated Secretory Phenotype (SASP) consisting of inflammatory cytokines, growth factors, and proteases is another characteristic feature of senescent cells (Malaquin N, Martinez A, Rodier F (2016). Experimental Gerontology. 82: 39-49). SASP is associated with many age-related diseases, including type 2 diabetes and atherosclerosis (Childs B G, et al. (2015)).

Accordingly, in certain embodiments, a composition comprises a therapeutically effective amount of an IL-17 inhibitory agent, a senolytic agent or a combination thereof. In certain embodiments, the composition further comprises a senomorphic agent. In certain embodiments, the senolytic agent, the senomorphic agent or the IL-17 inhibitory agent comprise: antibodies, antibody fragments, oligonucleotides, polynucleotides, antisense oligonucleotides, enzymes, gene editing agents, nucleases, peptides, polypeptides, small molecules, synthetic compounds, natural compounds or combinations thereof.

Senolytic agents are a class of drugs that selectively eliminate senescent cells or suppress the senescence-associated secretory phenotype (SASP) that drives sterile inflammation (senomorphics). In certain embodiments, senolytic agents comprise: dasatinib, quercetin, ABT-263 (navitoclax), ABT-737, piperlongumine (PL), fisetin, HSP90 inhibitors, A1331852, A1155463, ATTAC, BCL-X_{L} inhibitors, or combinations thereof. In certain embodiments, the senomorphic agent is an inhibitor of the function or expression of suppressor of expression senescent-associated secretory phenotype (SASP) factors, comprising rapamycin, NF-κB and JAK inhibitor, or combinations thereof.

In certain cases it may also be desirable to provide a booster to induce the immune response away from the T_{H}17 lineage. In those instances, the composition can comprise cytokines comprising: interleukin-4 (IL-4), interferon-gamma (IFN-γ), interleukin-12 (IL-12), (IL-27) or combinations thereof. In certain embodiments, these cytokines may be administered at alternative times and routes with regard to the compositions embodied herein.

In certain embodiments, a composition comprises a therapeutically effective amount of an IL-17 inhibitory agent and a senolytic agent. In certain embodiments, the senolytic agent or the IL-17 inhibitory agent comprise: antibodies, antibody fragments, oligonucleotides, polynucleotides, antisense oligonucleotides, enzymes, gene editing agents, nucleases, peptides, polypeptides, small molecules, synthetic compounds, natural compounds or combinations thereof. In certain embodiments, senolytic agents comprise: dasatinib, quercetin, ABT-263 (navitoclax), ABT-737, piperlongumine (PL), fisetin, HSP90 inhibitors, A1331852, A1155463, ATTAC, BCL-XL inhibitors, or combinations thereof. In certain embodiments, the composition further comprises a senomorphic agent, cytokines or a combination thereof. In certain embodiments, the cytokines comprises interleukin-4 (IL-4), interferon-gamma (IFN-γ), interleukin-12 (IL-12), (IL-27) or combinations thereof.

The pharmaceutical composition comprising the IL-17 inhibitory agents, senolytics etc., is administered in an effective amount. For example, an effective amount of the pharmaceutical composition is between about 1 μg/kg and 100 μg/kg, e.g., 1, 5, 10, 15, 20, 25, 30, 35, 40, 45, 50, 55, 60, 65, 70, 75, 80, 85, 90, 95, or 100 μg/kg. Alternatively, the composition is administered as a fixed dose or based on body surface area (i.e., per m²).

The pharmaceutical composition is administered at least one time per month, e.g., twice per month, once per week, twice per week, once per day, twice per day, every 8 hours, every 4 hours, every 2 hours, or every hour. Suitable modes of administration for the pharmaceutical composition include systemic administration, intravenous administration, local administration, subcutaneous administration, intramuscular administration, inhalation, and intraperitoneal administration.

In one aspect, compositions of the invention are administered systemically, for example, formulated in a pharmaceutically-acceptable buffer such as physiological saline. Preferable routes of administration include, for example, instillation into the bladder, subcutaneous, intravenous, intraperitoneal, intramuscular, intradermal injections that provide continuous, sustained, or effective levels of the composition in the patient. Treatment of human patients or other animals is carried out using a therapeutically effective amount of a therapeutic identified herein in a physiologically-acceptable carrier. Suitable carriers and their formulation are described, for example, in Remington's Pharmaceutical Sciences by E. W. Martin. The amount of the therapeutic agent to be administered varies depending upon the manner of administration, the age and body weight of the patient, and with the clinical symptoms of the FBR. Generally, amounts will be in the range of those used for other agents used in the treatment of other diseases associated with inflammatory responses.

### Inhibitory Agents

The compositions of the invention comprise therapeutically effective amounts of an IL-17 inhibitory agent, a senolytic agent or a combination thereof. Agents useful in the methods of the invention can be small molecules, but can also be enzymes and/or nucleic acid molecules, e.g., antisense, gene-editing agents, ribozyme, or RNA interference technology, e.g., siRNA molecules corresponding to a portion of the nucleotide sequence encoding IL-17.

In certain embodiments, an IL-17 inhibitory agent decreases expression of IL-17 by at least 1, 2, 3, 4, 5 7, 10, 15, 20, 25, 30, 40 50 60, 70, 80, 90 or 100 percent relative to the same test assay in the absence of the IL-17 inhibitory agent (control). The inhibition of IL-17 can be measured using commercially available kits or use of commercial screening services such as BPS Bioscience, San Diego, CA. For example, PATHHUNTER® Cell-based Assays for Human Interleukins, Eurofins DiscoverX Corporation, Fremont, Calif.

In some embodiments, the inhibitory agent comprises an antibody or fragment thereof (e.g. anti-IL-17 antibody), a binding protein, a polypeptide, or any combination thereof In some embodiments, the inhibitory agent comprises a small molecule. In some embodiments, the inhibitory agent comprises a nucleic acid molecule. In some embodiments, the oligonucleotides or polynucleotides comprise: ribonucleic acids (RNA), deoxyribonucleic acids (DNA), synthetic RNA or DNA sequences, modified RNA or DNA sequences, complementary DNA (cDNA), short guide RNA (sgRNA), a short interfering RNA (siRNA), a micro, interfering RNA (miRNA), a small, temporal RNA (stRNA), a short, hairpin RNA (shRNA), mRNA, nucleic acid sequences comprising one or more modified nucleobases or backbones, or combinations thereof.

Specific IL-17 inhibitors that can be utilized in the present methods and compositions include seckinumab, brodalumab and ixekizumab. See Wasilewska et al., Adv Dermatol Allergol 2016 33(11): 247-252; Dong et al., Cutis vol. 99, page 123-127 (February 2017). Additional IL-17 inhibitors are disclosed in U.S. Pat. No. 6,793,919.

Antisense polynucleotides may act by directly blocking translation by hybridizing to mRNA transcripts or degrading such transcripts of a gene e.g. IL-17, cytokines released by senescent cells, etc. The antisense molecule may be recombinantly made using at least one functional portion of a gene in the antisense orientation as a region downstream of a promoter in an expression vector. Chemically modified bases or linkages may be used to stabilize the antisense polynucleotide by reducing degradation or increasing half-life in the body (e.g., methyl phosphonates, phosphorothioate, peptide nucleic acids). The sequence of the antisense molecule may be complementary to the translation initiation site (e.g., between −10 and +10 of the target's nucleotide sequence).

siRNA refers to double-stranded RNA of at least 20-25 basepairs which mediates RNA interference (RNAi). Duplex siRNA corresponding to a target RNA may be formed by separate transcription of the strands, coupled transcription from a pair of promoters with opposing polarities, or annealing of a single RNA strand having an at least partially self-complementary sequence. Alternatively, duplexed oligoribonucleotides of at least about 21 to about 23 basepairs may be chemically synthesized (e.g., a duplex of 21 ribonucleotides with 3′ overhangs of two ribonucleotides) with some substitutions by modified bases being tolerated. Mismatches in the center of the siRNA sequence, however, abolishes interference. The region targeted by RNA interference should be transcribed, preferably as a coding region of the gene. Interference appears to be dependent on cellular factors (e.g., ribonuclease III) that cleave target RNA at sites 21 to 23 bases apart; the position of the cleavage site appears to be defined by the 5′ end of the guide siRNA rather than its 3′ end. Priming by a small amount of siRNA may trigger interference after amplification by an RNA-dependent RNA polymerase.

Nucleases: Any suitable nuclease system can be used including, for example, Argonaute family of endonucleases, clustered regularly interspaced short palindromic repeat (CRISPR) nucleases, zinc-finger nucleases (ZFNs), transcription activator-like effector nucleases (TALENs), meganucleases, other endo- or exo-nucleases, or combinations thereof See Schiffer, 2012, J Virol 88(17):8920-8936, incorporated by reference. In certain embodiments, the system is an Argonaute nuclease system.

CRISPR-Cas: In certain aspects, inhibition of, for example, IL-17 can be achieved by administration of inhibitory nucleic acids (e.g., dsRNAs, siRNAs, antisense oligonucleotides, etc.) directed to inhibit IL-17 or any other cytokine expression or activity. It is also contemplated that CRISPR-Cas (e.g., CRISPR-Cas9) methods can be used to excise and/or replace sections of genes encoding regulators of extracellular cytokine bioavailability Such methods can be performed upon the cells of a subject in vivo or ex vivo. Use of any combination of the above and/or other known inhibitor of IL-17 is also contemplated. The CRISPR-Cas system is known in the art. Non- limiting aspects of this system are described in U.S. Pat. No. 8,697,359, issued Apr. 15, 2014, the entire content of which is incorporated herein by reference.

Non-limiting examples of Cas proteins include Cas1, Cas1B, Cas2, Cas3, Cas4, Cas5, Cas6, Cas7, Cas8, Cas9 (also known as Csn1 and Csx12), Cas 10, Csy1, Csy2, Csy3, Cse1, Cse2, Csc1, Csc2, Csa5, Csn2, Csm2, Csm3, Csm4, Csm5, Csm6, Cmr1, Cmr3, Cmr4, Cmr5, Cmr6, Csb1, Csb2, Csb3, Csx17, Csx14, Csx1O, Csx16, CsaX, Csx3, Csx1, Csx15, Csf1, Csf2, Csf3, Csf4, homologs thereof, or modified versions thereof. These enzymes are known; for example, the amino acid sequence of S. pyogenes Cas9 protein may be found in the SwissProt database under accession number Q99ZW2. In some embodiments, the unmodified CRISPR enzyme has DNA cleavage activity, such as Cas9. In some embodiments, the CRISPR enzyme is Cas9, and may be Cas9 from S. pyogenes or S. pneumoniae. In some embodiments, the CRISPR enzyme directs cleavage of one or both strands at the location of a target sequence, such as within the target sequence and/or within the complement of the target sequence. In some embodiments, the CRISPR enzyme directs cleavage of one or both strands within about 1, 2, 3, 4, 5, 6, 7, 8, 9, 10, 15, 20, 25, 50, 100, 200, 500, or more base pairs from the first or last nucleotide of a target sequence. In some embodiments, a vector encodes a CRISPR enzyme that is mutated with respect to a corresponding wild-type enzyme such that the mutated CRISPR enzyme lacks the ability to cleave one or both strands of a target polynucleotide containing a target sequence. For example, an aspartate-to-alanine substitution (D10A) in the RuvC I catalytic domain of Cas9 from S. pyogenes converts Cas9 from a nuclease that cleaves both strands to a nickase (cleaves a single strand). Other examples of mutations that render Cas9 a nickase include, without limitation, H840A, N854A, and N863A. In aspects of the invention, nickases may be used for genome editing via homologous recombination.

A guide sequence is any polynucleotide sequence having sufficient complementarity with a target polynucleotide sequence to hybridize with the target sequence and direct sequence-specific binding of a CRISPR complex to the target sequence. In some embodiments, the degree of complementarity between a guide sequence and its corresponding target sequence, when optimally aligned using a suitable alignment algorithm, is about or more than about 50%, 60%, 75%, 80%, 85%, 90%, 95%, 97.5%, 99%, or more. Optimal alignment may be determined with the use of any suitable algorithm for aligning sequences, non-limiting example of which include the Smith-Waterman algorithm, the Needleman-Wunsch algorithm, algorithms based on the Burrows-Wheeler Transform (e.g., the Burrows Wheeler Aligner), ClustalW, Clustal X, BLAT, Novoalign (Novocraft Technologies, ELAND (Illumina, San Diego, Calif), SOAP (available at soap.genomics.org.cn), and Maq (available at maq.sourceforge.net). In some embodiments, a guide sequence is about or more than about 5, 10, 11, 12, 13, 14, 15, 16, 17, 18, 19, 20, 21, 22, 23, 24, 25, 26, 27, 28, 29, 30, 35, 40, 45, 50, 75, or more nucleotides in length. In some embodiments, a guide sequence is less than about 75, 50, 45, 40, 35, 30, 25, 20, 15, 12, or fewer nucleotides in length. The ability of a guide sequence to direct sequence-specific binding of a CRISPR complex to a target sequence may be assessed by any suitable assay. For example, the components of a CRISPR system sufficient to form a CRISPR complex, including the guide sequence to be tested, may be provided to a host cell having the corresponding target sequence, such as by transfection with vectors encoding the components of the CRISPR sequence, followed by an assessment of preferential cleavage within the target sequence, such as by Surveyor assay as described herein. Similarly, cleavage of a target polynucleotide sequence may be evaluated in a test tube by providing the target sequence, components of a CRISPR complex, including the guide sequence to be tested and a control guide sequence different from the test guide sequence, and comparing binding or rate of cleavage at the target sequence between the test and control guide sequence reactions. Other assays are possible, and will occur to those skilled in the art.

Argonautes: Argonautes are a family of endonucleases that use 5′ phosphorylated short single-stranded nucleic acids as guides to cleave targets (Swarts, D. C. et al. The evolutionary journey of Argonaute proteins. Nat. Struct. Mol. Biol. 21, 743-753 (2014)). Similar to Cas9, Argonautes have key roles in gene expression repression and defense against foreign nucleic acids (Swarts, D. C. et al. Nat. Struct. Mol. Biol. 21, 743-753 (2014); Makarova, K. S., et al. Biol. Direct 4, 29 (2009). Molloy, S. Nat. Rev. Microbiol. 11, 743 (2013); Vogel, J. Science 344, 972-973 (2014). Swarts, D.C. et al. Nature 507, 258-261 (2014); Olovnikov, I., et al. Mol. Cell 51, 594-605 (2013)). However, Argonautes differ from Cas9 in many ways (Swarts, D. C. et al. Nat. Struct. Mol. Biol. 21, 743-753 (2014)). Cas9 only exist in prokaryotes, whereas Argonautes are preserved through evolution and exist in virtually all organisms; although most Argonautes associate with single-stranded (ss)RNAs and have a central role in RNA silencing, some Argonautes bind ssDNAs and cleave target DNAs (Swarts, D. C. et al. Nature 507, 258-261 (2014); Swarts, D.C. et al. Nucleic Acids Res. 43, 5120-5129 (2015)). guide RNAs must have a 3′ RNA-RNA hybridization structure for correct Cas9 binding, whereas no specific consensus secondary structure of guides is required for Argonaute binding; whereas Cas9 can only cleave a target upstream of a PAM, there is no specific sequence on targets required for Argonaute. Once Argonaute and guides bind, they affect the physicochemical characteristics of each other and work as a whole with kinetic properties more typical of nucleic-acid-binding proteins (Salomon, W. E., et al. Cell 162, 84-95 (2015)).

If desired, the polynucleotides of the invention may also be used with a microdelivery vehicle such as cationic liposomes and adenoviral vectors. For a review of the procedures for liposome preparation, targeting and delivery of contents, see Mannino and Gould-Fogerite, BioTechniques, 6:682 (1988). See also, Felgner and Holm, Bethesda Res. Lab. Focus, 11(2):21 (1989) and Maurer, R. A., Bethesda Res. Lab. Focus, 11(2):25 (1989).

Antibodies: These molecules can be generated by any method known in the art. Antibodies can be generated not only against the desired molecule but also to the receptor thereby preventing engagement by the ligand, e.g. anti-IL-17 antibodies, anti-IL-17 receptor antibodies etc. Currently, one anti-IL17 biological agent is approved for the treatment—a fully human monoclonal antibody that targets IL-17A (secukinumab). Further clinical trials, including a humanized IgG4 specific for IL-17 (ixekizumab) and a fully human antibody that targets the IL-17 receptor A (brodalumab) (Wasilewska, Agnieszka et al.. “Interleukin-17 inhibitors. A new era in treatment of psoriasis and other skin diseases” Postepy Dermatol Alergol. 2016 August; 33(4): 247-252). Numerous pipeline biologics, include risankizumab, guselkumab, tildrakizumab, and bimekizumab. Bimekizumab is a humanized IgG1 monoclonal antibody which uniquely neutralizes both IL-17A and IL-17F (Glatt S, Baeten D, Baker T, et al. Annals of the Rheumatic Diseases 2018; 77:523-532).

The term “antibody” means not only intact antibody molecules, but also fragments of antibody molecules that retain immunogen-binding ability. Such fragments are also well known in the art and are regularly employed both in vitro and in vivo. Accordingly, as used herein, the term “antibody” means not only intact immunoglobulin molecules but also the well-known active fragments F(ab′)₂, and Fab. F(ab′)₂, and Fab fragments that lack the Fc fragment of intact antibody, clear more rapidly from the circulation, and may have less non-specific tissue binding of an intact antibody (Wahl et al., J. Nucl. Med. 24:316-325 (1983). The antibodies of the invention comprise whole native antibodies, bispecific antibodies; chimeric antibodies; Fab, Fab′, single chain V region fragments (scFv), fusion polypeptides, and unconventional antibodies. As used herein, the term “single-chain variable fragment” or “scFv” is a fusion protein of the variable regions of the heavy (VH) and light chains (VL) of an immunoglobulin covalently linked to form a VH: :VL heterodimer. The heavy (VH) and light chains (VL) are either joined directly or joined by a peptide-encoding linker (e.g., 10, 15, 20, 25 amino acids), which connects the N-terminus of the VH with the C-terminus of the VL, or the C-terminus of the VH with the N-terminus of the VL. In some embodiments, the linker includes glycine for flexibility, and serine or threonine for solubility. scFv proteins retain the specificity of the original immunoglobulin. Single chain Fv antibodies can be expressed as described by Huston, et al. (Proc. Nat. Acad. Sci. USA, 85:5879-5883, 1988). See, also, U.S. Pat. Nos. 5,091,513, 5,132,405 and 4,956,778; and U.S. Patent Publication Nos. 20050196754 and 20050196754. Antagonistic scFvs having inhibitory activity have been described (see, e.g., Zhao et al., Hyrbidoma (Larchmt) 2008 27(6):455-51; Peter et al., J Cachexia Sarcopenia Muscle 2012 Aug. 12; Shieh et al., J Imunol 2009 183(4):2277-85; Giomarelli et al., Thromb Haemost 2007 97(6):955-63; Fife et al., J Clin Invst 2006 116(8):2252-61; Brocks et al., Immunotechnology 1997 3(3): 173-84; Moosmayer et al., Ther Immunol 1995 2(10:31-40). Agonistic scFvs having stimulatory activity have been described (see, e.g., Peter et al., J Bioi Chem 2003 25278(38):36740-7; Xie et al., Nat Biotech 1997 15(8):768-71; Ledbetter et al., Crit Rev Immunol1997 17(5-6):427-55; Ho et al., BioChim Biophys Acta 2003 1638(3):257-66).

### Formulation of Pharmaceutical Compositions

The administration of the compositions of the invention for the treatment of an FBR e is by any suitable means that results in a concentration of the therapeutic that, combined with other components, is effective in ameliorating, reducing, or stabilizing said FBR. The compositions may be contained in any appropriate amount in any suitable carrier substance, and is generally present in an amount of 1-95% by weight of the total weight of the composition. The composition may be provided in a dosage form that is suitable for parenteral (e.g., subcutaneous, intravenous, intramuscular, intravesicular, intratumoral or intraperitoneal) administration route. For example, the pharmaceutical compositions are formulated according to conventional pharmaceutical practice (see, e.g., Remington: The Science and Practice of Pharmacy (20th ed.), ed. A. R. Gennaro, Lippincott Williams & Wilkins, 2000 and Encyclopedia of Pharmaceutical Technology, eds. J. Swarbrick and J. C. Boylan, 1988-1999, Marcel Dekker, New York).

Human dosage amounts are initially determined by extrapolating from the amount of compound used in mice or non-human primates, as a skilled artisan recognizes it is routine in the art to modify the dosage for humans compared to animal models. For example, the dosage may vary from between about 1 μg compound/kg body weight to about 5000 mg compound/kg body weight; or from about 5 mg/kg body weight to about 4,000 mg/kg body weight or from about 10 mg/kg body weight to about 3,000 mg/kg body weight; or from about 50 mg/kg body weight to about 2000 mg/kg body weight; or from about 100 mg/kg body weight to about 1000 mg/kg body weight; or from about 150 mg/kg body weight to about 500 mg/kg body weight. For example, the dose is about 1, 5, 10, 25, 50, 75, 100, 150, 200, 250, 300, 350, 400, 450, 500, 550, 600, 650, 700, 750, 800, 850, 900, 950, 1,000, 1,050, 1,100, 1,150, 1,200, 1,250, 1,300, 1,350, 1,400, 1,450, 1,500, 1,600, 1,700, 1,800, 1,900, 2,000, 2,500, 3,000, 3,500, 4,000, 4,500, or 5,000 mg/kg body weight. Alternatively, doses are in the range of about 5 mg compound/Kg body weight to about 20 mg compound/kg body weight. In another example, the doses are about 8, 10, 12, 14, 16 or 18 mg/kg body weight. Preferably, the fusion protein complex is administered at 0.5 mg/kg-about 10 mg/kg (e.g., 0.5, 1, 3, 5, 10 mg/kg). Of course, this dosage amount may be adjusted upward or downward, as is routinely done in such treatment protocols, depending on the results of the initial clinical trials and the needs of a particular patient.

Pharmaceutical compositions are formulated with appropriate excipients into a pharmaceutical composition that, upon administration, releases the therapeutic in a controlled manner. Examples include single or multiple unit tablet or capsule compositions, oil solutions, suspensions, emulsions, microcapsules, microspheres, molecular complexes, nanoparticles, patches, and liposomes. Preferably, the fusion protein complex is formulated in an excipient suitable for parenteral administration.

### Parenteral Compositions

The pharmaceutical composition are administered parenterally by injection, infusion, or implantation (subcutaneous, intravenous, intramuscular, intravesicular, intraperitoneal) in dosage forms, formulations, or via suitable delivery devices or implants containing conventional, non-toxic pharmaceutically acceptable carriers and adjuvants. The formulation and preparation of such compositions are well known to those skilled in the art of pharmaceutical formulation. Formulations can be found in Remington: The Science and Practice of Pharmacy, supra.

Compositions for parenteral use are provided in unit dosage forms (e.g., in single-dose ampoules). Alternatively, the composition is provided in vials containing several doses and in which a suitable preservative may be added (see below). The composition is in the form of a solution, a suspension, an emulsion, an infusion device, or a delivery device for implantation, or it is presented as a dry powder to be reconstituted with water or another suitable vehicle before use. Apart from the active agent that reduces or ameliorates a neoplasia, infectious or autoimmune disease, the composition includes suitable parenterally acceptable carriers and/or excipients. The active therapeutic agent(s) may be incorporated into microspheres, microcapsules, nanoparticles, liposomes for controlled release. Furthermore, the composition may include suspending, solubilizing, stabilizing, pH-adjusting agents, tonicity adjusting agents, and/or dispersing, agents.

The nucleic acid inhibitory agents of the invention can be delivered to an appropriate cell of a subject. This can be achieved by, for example, the use of a polymeric, biodegradable microparticle or microcapsule delivery vehicle, sized to optimize phagocytosis by phagocytic cells such as macrophages. For example, PLGA (poly-lacto-co-glycolide) microparticles approximately 1-10 μm in diameter can be used. The polynucleotide is encapsulated in these microparticles, which are taken up by macrophages and gradually biodegraded within the cell, thereby releasing the polynucleotide. Once released, the DNA is expressed within the cell. A second type of microparticle is intended not to be taken up directly by cells, but rather to serve primarily as a slow-release reservoir of nucleic acid that is taken up by cells only upon release from the micro-particle through biodegradation. These polymeric particles should therefore be large enough to preclude phagocytosis (i.e., larger than 5 μm and preferably larger than 20 μm). Another way to achieve uptake of the nucleic acid is using liposomes, prepared by standard methods. The nucleic acids can be incorporated alone into these delivery vehicles or co-incorporated with tissue-specific antibodies. Alternatively, one can prepare a molecular complex composed of a plasmid or other vector attached to poly-L-lysine by electrostatic or covalent forces. Poly-L-lysine binds to a ligand that can bind to a receptor on target cells. Delivery of “naked DNA” (i.e., without a delivery vehicle) to an intramuscular, intradermal, or subcutaneous site, is another means to achieve in vivo expression. In the relevant polynucleotides (e.g., expression vectors) the nucleic acid sequence encoding the an isolated nucleic acid sequence comprising a sequence encoding a CRISPR-associated endonuclease and a guide RNA is operatively linked to a promoter or enhancer-promoter combination. Promoters and enhancers are described above.

In some embodiments, the compositions of the invention can be formulated as a nanoparticle, for example, nanoparticles comprised of a core of high molecular weight linear polyethylenimine (LPEI) complexed with DNA and surrounded by a shell of polyethyleneglycol-modified (PEGylated) low molecular weight LPEI.

As indicated above, the pharmaceutical compositions comprising a fusion protein complex of the invention may be in a form suitable for sterile injection. To prepare such a composition, the suitable active therapeutic(s) are dissolved or suspended in a parenterally acceptable liquid vehicle. Among acceptable vehicles and solvents that may be employed are water, water adjusted to a suitable pH by addition of an appropriate amount of hydrochloric acid, sodium hydroxide or a suitable buffer, 1,3-butanediol, Ringer's solution, and isotonic sodium chloride solution and dextrose solution. The aqueous formulation may also contain one or more preservatives (e.g., methyl, ethyl, or n-propyl p-hydroxybenzoate). In cases where one of the compounds is only sparingly or slightly soluble in water, a dissolution enhancing or solubilizing agent can be added, or the solvent may include 10-60% w/w of propylene glycol.

The present invention provides methods of preventing or treating an FBR which comprise administering a therapeutically effective amount of a pharmaceutical composition comprising a compound of the formulae herein to a subject (e.g., a mammal such as a human). Thus, one embodiment is a method of In certain embodiments, a method of preventing or inhibiting a foreign body response(FBR) in a subject, comprises administering to the subject a pharmaceutical composition comprising a therapeutically effective amount of an agent which inhibits interleukin-17 (IL-17) activity or function. In certain embodiments, the agent inhibiting IL-17 inhibits IL-17-producing γδ T cells and CD4⁺ T_{H}17 cells in tissue surrounding the foreign body. In certain embodiments, the administration of the agent inhibiting IL-17 results in reduction of expression of p16, p21, IL-17, type I collagen, S100a4 or combinations thereof. In certain embodiments, the method further comprises administering a senolytic agent, a senomorphic agent, an inhibitor of interleukin-6 (IL-6), an inhibitor of interleukin 1β (IL-1β), an inhibitor of tumor necrosis factor α (TNFα), an inhibitor of interleukin-21 (IL-21), an inhibitor of interleukin-23 (IL-23) or combinations thereof. The senolytic agent selectively lyses or selectively kills senescent cells. In certain embodiments, the agent inhibiting IL-17 expression or function and the senolytic agent are administered concomitantly or at different times.

In certain embodiments, the senolytic agent or the agent inhibiting IL-17 comprise: antibodies, antibody fragments, oligonucleotides, polynucleotides, antisense oligonucleotides, enzymes, gene editing agents, nucleases, peptides, polypeptides, small molecules, synthetic compounds, natural compounds or combinations thereof.

In certain embodiments, the method of preventing or inhibiting a T helper 17 (T_{H}17) cellular response in a subject, comprises administering to the subject a pharmaceutical composition comprising a therapeutically effective amount of an inhibitor of interleukin-17 (IL-17) activity or function. In certain embodiments, the inhibitor of IL-17 inhibits expression of p16, p21, IL-17, type I collagen, S100a4 or combinations thereof. In certain embodiments, the method further comprises administering a senolytic agent, an inhibitor of interleukin-6 (IL-6), an inhibitor of interleukin 1β(IL-1β) or combinations thereof. In certain embodiments, cytokines which reduce T_{H}17 cells can also be administered. The cytokines comprise interleukin-4 (IL-4), interferon-gamma (IFN-γ), interleukin-12 (IL-12), (IL-27) or combinations thereof.

The methods herein include administering to the subject (including a subject identified as in need of such treatment) an effective amount of a compound described herein, or a composition described herein to produce such effect. Identifying a subject in need of such treatment can be in the judgment of a subject or a health care professional and can be subjective (e.g. opinion) or objective (e.g. measurable by a test or diagnostic method).

The therapeutic methods of the invention (which include prophylactic treatment) in general comprise administration of a therapeutically effective amount of the compounds herein, such as a compound of the formulae herein to a subject (e.g., animal, human) in need thereof, including a mammal, particularly a human. Such treatment will be suitably administered to subjects, particularly humans, suffering from, having, susceptible to, or at risk an FBR, or symptom thereof. Determination of those subjects “at risk” can be made by any objective or subjective determination by a diagnostic test or opinion of a subject or health care provider (e.g., genetic test, enzyme or protein marker, Marker (as defined herein), family history, and the like). The fusion protein complexes of the invention may be used in the treatment of any other disorders in which an increase in an immune response is desired.

The invention also provides a method of monitoring treatment progress. The method includes the step of determining a level of diagnostic marker (Marker) (e.g., any target delineated herein modulated by a compound herein, a protein or indicator thereof, etc.) or diagnostic measurement (e.g., screen, assay) in a subject suffering from or susceptible to an FBR in which the subject has been administered a therapeutic amount of a compound herein sufficient to treat the disease or symptoms thereof. The level of Marker determined in the method can be compared to known levels of Marker in either healthy normal controls or in other afflicted patients to establish the subject's disease status. In some cases, a second level of Marker in the subject is determined at a time point later than the determination of the first level, and the two levels are compared to monitor the course of disease or the efficacy of the therapy. In certain aspects, a pre-treatment level of Marker in the subject is determined prior to beginning treatment according to this invention; this pre-treatment level of Marker can then be compared to the level of Marker in the subject after the treatment commences, to determine the efficacy of the treatment.

### Combination Therapies

Optionally, the compositions of the invention is administered in combination with any other standard therapy; such methods are known to the skilled artisan and described in Remington's Pharmaceutical Sciences by E. W. Martin. If desired, the compositions of the invention is administered in combination with any conventional anti-inflammatory, anti-fibrosis, autoimmune treatments.

### Kits or Pharmaceutical Systems

Pharmaceutical compositions comprising the agents of the invention may be assembled into kits or pharmaceutical systems for use in ameliorating a neoplasia, infectious or autoimmune disease. Kits or pharmaceutical systems according to this aspect of the invention comprise a carrier means, such as a box, carton, tube, having in close confinement therein one or more container means, such as vials, tubes, ampoules, bottles, and the like. The kits or pharmaceutical systems of the invention may also comprise associated instructions for using the fusion protein complex of the invention.

### EXAMPLES

### Materials and Methods:

Study Design

We aimed to investigate the mechanisms underlying senescence and IL17 driven FBR. We performed detailed analysis of the immune cells in multiple tissue samples surrounding implants removed from patients undergoing breast implant exchange surgery using flow cytometry and immunofluorescence staining. To further investigate the role of IL17 in implant-associated fibrosis suggested by our clinical findings, we implanted synthetic materials in C57BL/6 mice using a volumetric muscle loss model. Materials were placed both subcutaneous or in a muscle wound to mimic surgical implantation and tissue trauma. Poly(caprolactone) (PCL), polyethylene glycol (PEG), and silicone, were used as implant materials. To evaluate if fibrosis associated with the FBR to biomaterials was IL17 dependent, we compared the implant response in IL17A^{-/-} and IL17RA^{-/-} mice with WT mice. We associated the role of senescence to FBR induced by implantation of biomaterials, both human and murine. We further demonstrated a connection and positive feedback between IL17 responses and cellular senescence in modulating the outcome of FBR via administration of senolytic and/or IL17 neutralizing antibodies.

Clinical Samples

Tissue was acquired from patients undergoing implant exchange or replacement surgeries (n=12, JHU IRB exemption IRB00088842). For each patient, up to 4 tissue sections were profiled including the left anterior, left posterior, right anterior, and right posterior with respect to the anatomical position of the implant (FIG. S1A). Each section was weighed and 0.25 g of tissue was dissected for histology. Remaining tissue was analyzed depending on the available quantity; qRT-PCR assay (<1 g), flow cytometry (1-2 g), or both (>2 g). In this study, 8 samples were utilized for qRT-PCR and 5 samples were used for flow cytometry from different individuals (n=12). Peri-implant samples included tissues surrounding implants with both smooth and textured surface properties. All implants had a silicone shell and were either temporary tissue expanders filled with saline or air or permanent implants filled with silicone or saline. Average patient age was 56 years old (range of 41-70 years old) and the average implant residence time was 41 months (range of 1-360 months).

Surgical Procedures and Implantation

All animal procedures were performed in accordance with an approved JHU IACUC protocol. Female mice were aged six to eight weeks-old. Several strains were utilized, including wild type C57BL/6j (Jackson Laboratories, Stock #00064), Rag2/OT-II (Taconic Stock #11490), IL17A−/− and IL17RA−/− (courtesy of Dr. Yoichiro Iwakura, University of Tokyo, Tokyo, Japan and Dr. Tomas Mustelin, Amgen, Seattle, respectively). Defects in muscle for material implantation were created as previously described (68). The resulting bilateral muscle defects were filled with 30 mg of a synthetic material. Synthetic materials tested include PCL (particulate, Mn=50000 g/mol, mean particle size<600 μm, Polysciences), ultrahigh molecular weight PE (particulate, Mn=5000 k g/mol, mean particle size=150 μm, Goodfellow), medical graded Silicone (sheet, Summit Medical), PEG hydrogels. PEG hydrogels were made with polyethylene glycol-diacrylate (PEG-DA, Mn=3400). PEG-DA (Polysciences) in PBS at 10% w/v concentration was mixed with photoinitiator (Irgacure 2959 solubilized to 10% w/v in 70% ethanol) to a final concentration of 0.05%. A 50 μL volume solution was cast onto each well of a flat bottom 96 wells plate. The solution was photocrosslinked via UV light for 30 mins to form a hydrogel sheet. Both silicone and PEG hydrogels were morselized into particles with a scalpel and placed into the defect. Control implants were injected with 50 μL of PBS (as a no implant control). All materials were UV sterilized prior to use. Directly after surgery, mice were given subcutaneous carprofen (Rimadyl®, Zoetis) at 5 mg/kg for pain relief. Mice were euthanized, and their implants were extracted at different time points: 1, 3, 6, or 12-weeks post-surgery. Additional modes of implantation were subcutaneous and intra-muscular injection. Briefly, synthetic materials were implanted subcutaneously (30 mg) on both flanks of the mice, or injected directly into the quadricep muscles through a 16 G syringe (5 mg of materials).

Quantitative Real-Time Polymerase Chain Reaction (qRT-PCR) Assay

Total RNA was isolated from whole tissue using TRIzol reagent and Qiagen's RNeasy kits. qRT-PCR was performed using Power SYBR Green Master Mix (Applied Biosystems) or TaqMan Gene Expression Master Mix (Applied Biosystems) according to manufacturer's instructions. Briefly, 2 μg of total RNA was used to synthesize cDNA using Superscript IV VILO Master Mix (ThermoFisher Scientific). The cDNA concentration was set to 50 ng/well (in a total volume of 20 μL PCR reaction). The qRT-PCR reactions were performed on the StepOne Plus Real-Time PCR System (ThermoFisher Scientific) using manufacturer recommended settings for quantitative and relative expression. Primers used for qRT-PCR are listed in Tables 1, 2, and 3.

Flow Cytometry

Tissue samples were obtained by cutting the quadriceps femoris muscle from the hip to the knee. Tissues were finely diced and digested for 45 min at 37oC with 1.67 Wünsch U/ml Liberase TL (Roche Diagnostics) and 0.2 mg/ml DNase I (Roche Diagnostics) in RPMI 1640 medium (Gibco). The digested tissues were ground through 100 μm cell strainers (ThermoFisher Scientific) with excess RPMI, and then washed twice with 1X DPBS. Percoll (GE Healthcare) density gradient centrifugation was used to enrich the leukocyte fraction and remove debris from the muscle samples. For intracellular staining, cells were stimulated for 4 hours with Cell Stimulation Cocktail (plus protein transport inhibitors) (eBioscience) diluted in complete culture media (IMDM supplemented with 5% fetal bovine serum). Cells were washed and surface stained, followed by fixation/permeabilization (Cytofix/Cytoperm, BD), and then stained for intracellular markers. Flow cytometry was performed using Attune NxT Flow Cytometer (ThermoFisher Scientific). The enriched cells were washed, and stained with the antibody panels listed in Table 4 and 5.

Immunofluorescence

IL17 and p16 was stained using tyramide signal amplification (TSA) method with Opal-570 (PerkinElmer, Cat #FP1488001KT) and Opal-650 (PerkinElmer, Cat #FP1496001KT) respectively. Briefly, after blocking with bovine serum albumin, the first primary antibody was incubated at room temperature for 30 mins, followed by 10 mins of incubation with HRP polymer conjugated secondary antibody, and 10 mins of Opal-650. Unbound antibodies were stripped by microwaving in citrate buffer for 15 mins to allow introduction of the next primary antibody (with different Opal dyes). Slides were then counterstained with DAPI for 5 mins before being mounted using DAKO mounting medium (Agilent, Cat #S302380-2). Imaging of the histological samples was performed on a Zeiss Axio Imager A2 and Zeiss AxioVision software ver. 4.2. Subsequent images were stitched together using ImageJ2 software.

IL17 neutralization, IL6 blocking antibody and senolytic treatment

Mice received 100 μl intraperitoneal (IP) injections of anti-IL17A (100 μg/ml, Cat #P13705.15) and anti-IL17F (100 μg/ml, Cat #P56220.17) (provided by Amgen) or isotype control (rat IgG2a, ThermoFisher Scientific, Cat #02-9688) every other day for a week. IL6 blocking antibody was administrated at 100 μg/ml (BioXCell, clone MP5-20F3) per mouse per day for 5 consecutive days. For senolytic treatment, mice received IP and intramuscular (IM) injections of Navitoclax (100 mg/kg, Selleckchem, Cat #HY-10087) for 5 consecutive days or vehicle control (5% DMSO, 3% Tween 80 in PBS). Co-administration of anti-IL17A/F and senolytic treatment were also evaluated. All mice received treatments at 4-weeks post-surgery for a total of 5 injections (1 injection per day per mouse), and were harvested in the following 2 weeks.

Statistical Analysis

The nCounter differential gene expression system was used to assess readable transcripts via the nanoString PanCancer Immune Profiling Panel. We utilized the nSolver software (version 3.0, nanoString Technologies, Inc.). All analyses of qRT-PCR data utilized the Livak Method, wherein ΔΔCt values are calculated, and then reported as relative quantification values calculated by 2-ΔCt (69). Treatment data points are normalized to either healthy or saline treated controls. β2M was utilized as the reference gene. In the flow cytometry reports, the total number of cytokine-secreting cells were computed using FlowJo software, and are displayed as the means±SD. Two-way ANOVAs were performed using GraphPad Prism v6, with statistical significance designated at p≥0.05. Linear regression was used for the analysis of association between gene expression levels in human samples.

Bone Marrow Chimera

Three million bone marrow cells from each donor mouse (CD45.1 WT mice (Jackson Laboratory, Stock #002014) and CD45.2 OTII-RagKO mice (Taconic, Stock #11490)) were injected intravenously into lethally irradiated (1200 cGy: twice at 600 cGy at 4 hours intervals) to 6-weeks old recipients. Bone marrow reconstituted mice were rested for 3 months prior to the VML procedure.

Histopathology

Tissues were harvested and fixed in 10% neutral buffered formalin for 24 hours before step-wise dehydration in EtOH, cleared with xylenes, and embedded in paraffin. Samples were sectioned as 7 μm slices using a Leica RM2255 microtome. Samples were stained for histopathological examination using Masson's trichrome (Sigma-Aldrich), hematoxylin and eosin (Sigma- Aldrich), and picrosirius red (Abcam) stains according to standard manufacturer protocols.

Treadmill Testing

Prior to testing, mice were trained on a treadmill apparatus at 5 m/min for 5 mins. Mice were run to exhaustion starting at the speed of 5 m/min, with 1 m/min speed increase every minute. Exhaustion was determined when the mouse stopped running, and stayed on the pulsed shock grid for a continuous 30 seconds. All mice were evaluated at 3, 6, and 12-weeks post-injury.

Antibody Titer

PCL particles were dissolved in chloroform to coat the bottom of 96-well plates (20 mg/ml). Sera collected 3, 6, and 12-weeks post-injury were serially diluted (in the range of 1:50 to 1:102400) in ELISA Assay Diluent (BioLegend, Cat #421203). Prior to loading, the PCL-coated plate was blocked with the assay diluent for 1 hour. After blocking, each dilution of the serum sample was loaded into the plate and incubated for 2 hours. After washing, biotin anti-mouse IgG1, or IgM, or IgA was added to capture the bound antibody for 1 hour, followed by washing. Streptavidin solution was added to the wells and incubated for 30 mins. TMB Substrate was used for HRP detection, and stopped with H2SO4. Absorbance was read at 450 nm and 570 nm (background control).

T_{H}17 In-Vitro Skewing Assay

Naïve CD4+ T cells were isolated from mouse spleens and lymph nodes using the Miltenyi Biotec Naïve CD4+ Isolation Kit according to manufacturer's protocol. Cells were differentiated using CellXVivo Mouse TH17 Cell Differentiation Kit (R&D Systems, Cat #CDK017) according to manufacturer's protocol. Cells were cultured for 5 days (refreshed the differentiation media at day 3). On day 5, cells were stimulated with Cell Stimulation Cocktail for 4 hours prior to cytokine staining, and examined using flow cytometry.

Cell Proliferation Assays

Primary Rechallenge Experiments

Mice were given an initial challenge of PCL or saline control treatment, locally delivered into the VML injury space (priming). The mice were then challenged/rechallenged with PCL by subcutaneous implant 1-week after the initial injury and treatment. In brief, following anesthesia, both flanks of mice were shaved, disinfected with 70% ethanol. A sterile blade was used to make a 5 mm incision through the skin only. Sterile forceps were used to sperate the skin to create approximately 4×4 cm subcutaneous pocket. Approximately 10 mg of PCL materials were placed inside the pocket on each side. The skin was then apposed and stapled. At 2-weeks post—VML, we harvested the cells infiltrating the SubQ implants and assessed their gene expression by qRT-PCR.splenocytes were isolated from OT-II transgenic mice. Cells were labeled with Cell Trace Violet Cell Proliferation Kit (Invitrogen) according to manufacturer's protocol. Three million cells/well were seeded into 6-well plates and were grown in RPMI supplemented with 10% fetal calf serum (Gibco), nonessential amino acids, sodium pyruvate (Invitrogen), and penicillin-streptomycin (Invitrogen). At the same time, ovalbumin (OVA) and biomaterials (PCL or PE—10 mg/well) were introduced into culture. After 48 hours, cells were harvested and analyzed using flow cytometry.

### Example 1

### IL-17 from T_{H}17, γδ and innate Lymphoid Cells and Cellular Senescence Regulate the Foreign Body Response

Innate lymphocytes (ILCs), γδ⁺ and CD4⁺ T cells were identified as the primary sources of IL-17 that promotes a fibrotic response to biomaterials. The interplay between IL-17 and cellular senescence was established as a mechanism linking the chronic immune response to synthetic implants to excessive fibrosis, a novel concept that introduces immune-stromal interactions as therapeutic target.

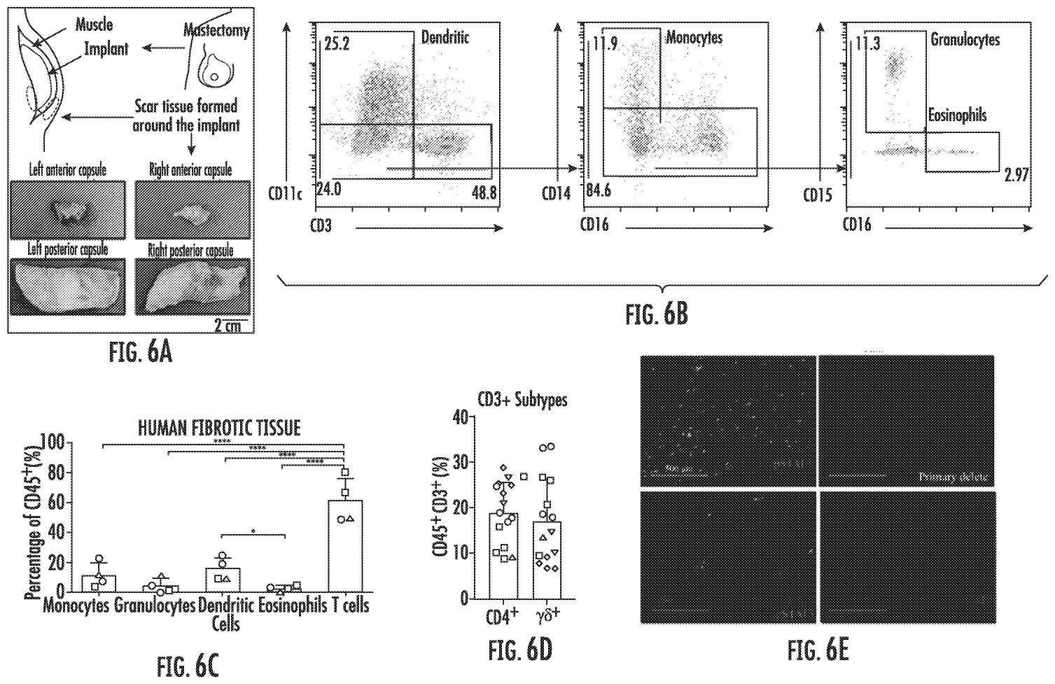

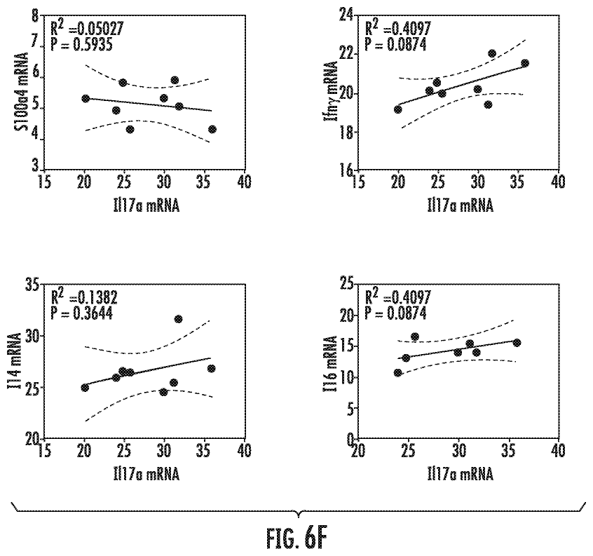

Interleukin 17 secreted by T cells is associated with fibrosis in tissue surrounding human breast implants: Breast implants suffer from fibrosis that causes capsular contraction that frequently necessitates removal and replacement. A detailed analysis of the immune cells was performed in tissues surrounding implants removed from patients undergoing breast implant exchange surgery. All implants had a silicone shell and were either temporary tissue expanders or permanent implants filled with silicone or saline. Implant samples included both normal and textured surface properties. Implants were originally placed adjacent to either adipose or muscle tissue depending on pre- or post-pectoral implantation site. For each patient, up to 4 tissue sections were profiled, including left anterior, left posterior, right anterior, and right posterior with respect to the anatomical position of the implant (FIG. 6A).

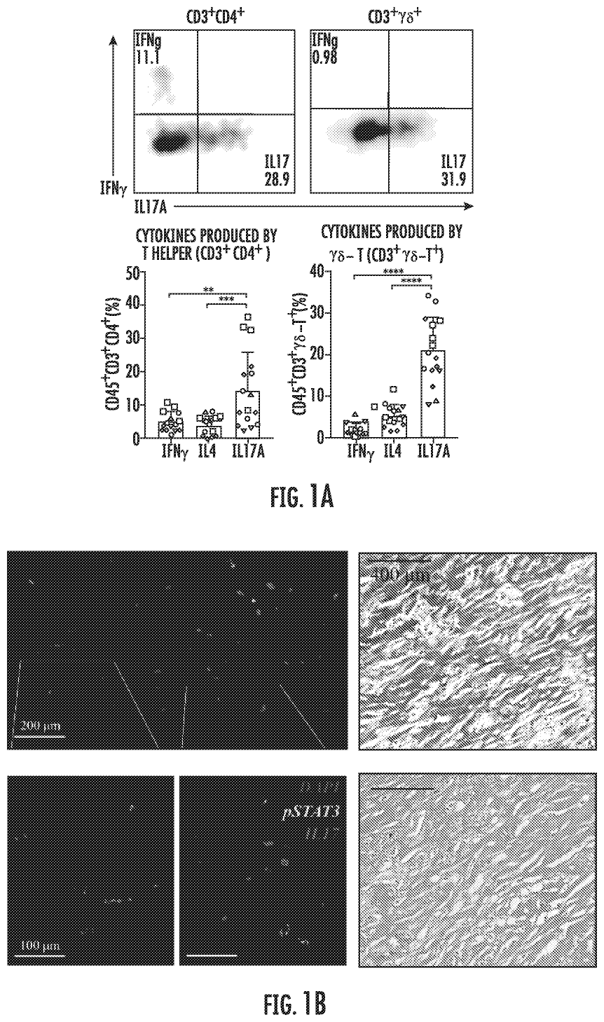

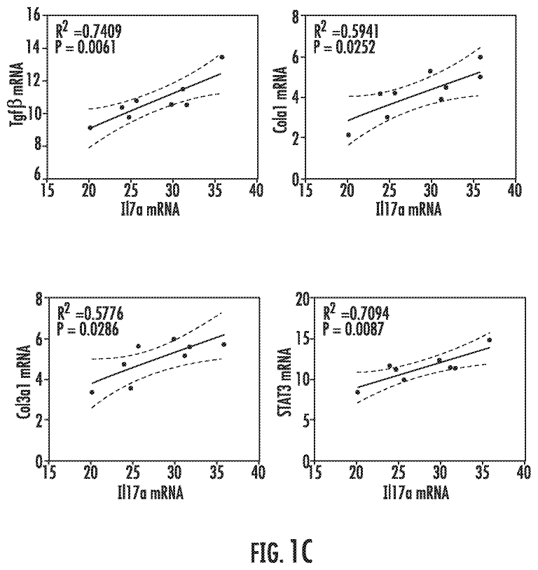

Multiparametric flow cytometry of infiltrating CD45+ leukocytes revealed the presence of large numbers of CD3+ T cells in addition to myeloid populations of mononuclear phagocytes, dendritic cells, eosinophils, and granulocytes (FIG. 6B and FIG. 6C). Intracellular cytokine staining of CD4+ T cells revealed significantly higher numbers of IL17 producing cells (TH17) compared to interferon gamma (IFNγ) (TH1) and IL4 (TH2)-producing cells in the tissue surrounding the implants (p<0.001) (FIG. 1A). γδ+ T cells (CD45+CD3+γδ+) represented a high proportion of the total CD3+ cells (Mean±SD: 16.97%±8.98%, FIG. 6D) around the implants and expressed IL17 similar to the CD4+ T cells. Immunofluorescence confirmed the presence of IL17 with concomitant nuclear staining of phosphorylated signal transducer and activator of transcription 3 (pSTAT3) that is essential for IL17 expression (FIG. 1B, FIG. 6E) (27). Overall, these results support a consistent type 17 immune response (also termed type 3 immunity (28)) to human silicone implants independent of the surface properties and implantation site, with both γδ+ and CD4+ T cells serving as the primary contributors to IL17 production.

To investigate if the TH17/IL17 immune signature contributed to the fibrosis observed in human tissue surrounding the implants (FIG. 1B), we evaluated mRNA expression of collagen I and III, transforming growth factor β (Tgfβ), and the fibroblast specific protein S100a4 (FIG. 1C, FIG. 6F) (29-31). Expression of Il17 positively correlated with expression of collagen I (R2=0.5941, p=0.0252) and collagen III (R2=0.5776, p=0.0286). The expression of Il17 also correlated with the fibrosis-related growth factor Tgfβ (R2=0.7409, p=0.0061). There was also a significant correlation between Il17 expression and STAT3 (R2=0.7094, p=0.0087), indicating a STAT3-dependent mechanism, further supporting the relevance of TH17 T cells in the FBR in patients. Together, these results suggest that the IL17 produced by γδ+ and CD4+ T cells may contribute to the regulation of fibrogenesis in response to the breast implants.

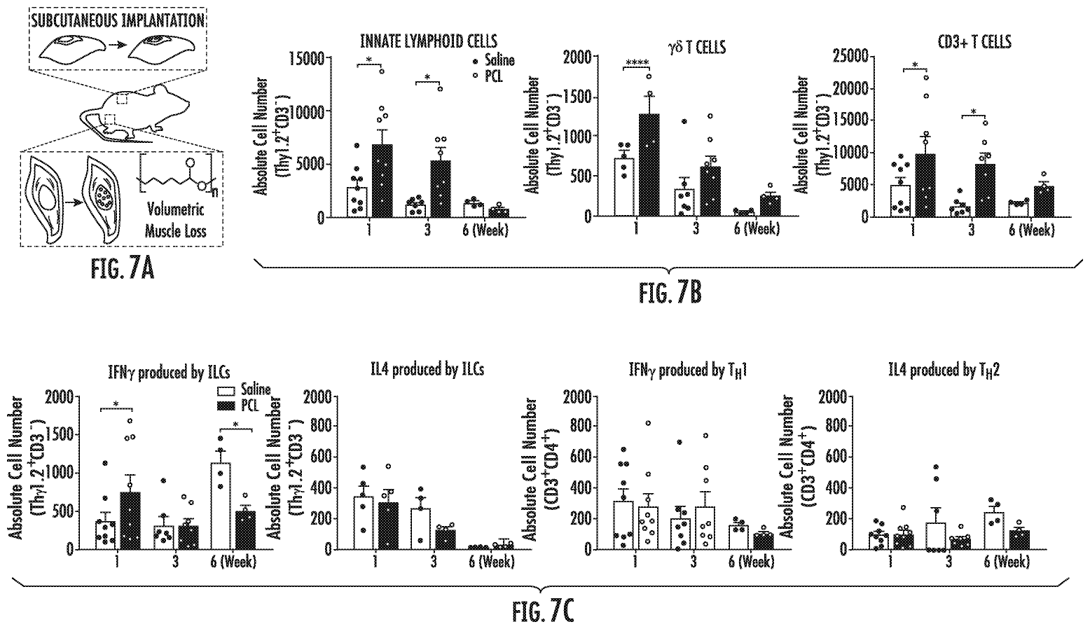

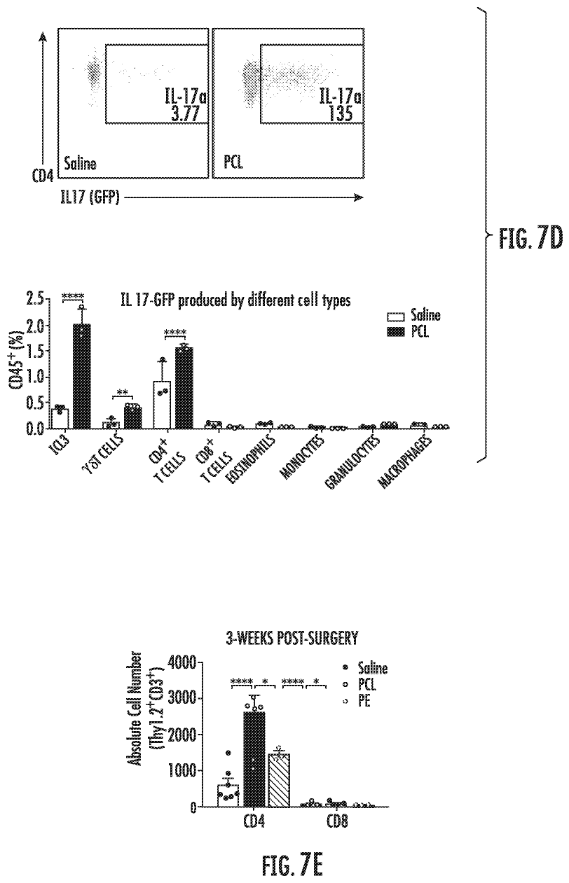

Innate and adaptive lymphocytes sequentially contribute to chronic IL-17 production in response to synthetic materials: To further investigate the role of IL17 in implant-associated fibrosis suggested by our clinical findings, and understand the mechanism linking the type 17 immune response to the FBR, we implanted synthetic materials in C57BL/6 mice. Materials were placed both subcutaneous or in a muscle wound to mimic surgical implantation and tissue trauma (FIG. 7A). Poly(caprolactone), PCL, was used as the primary model synthetic material since it induces a robust inflammatory response and is also a component of clinical implants (32). Results were validated with additional clinically-relevant synthetic materials including polyethylene (PE), polyethylene glycol (PEG), and silicone.

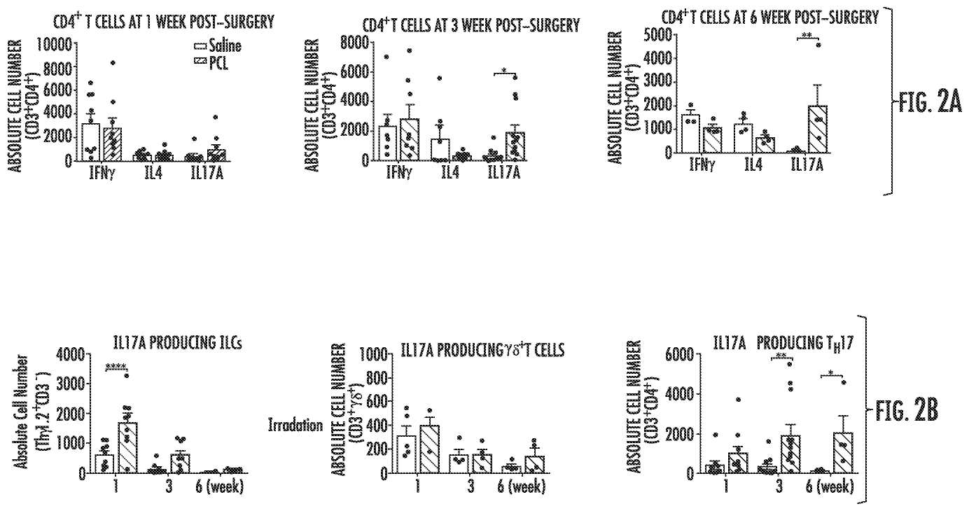

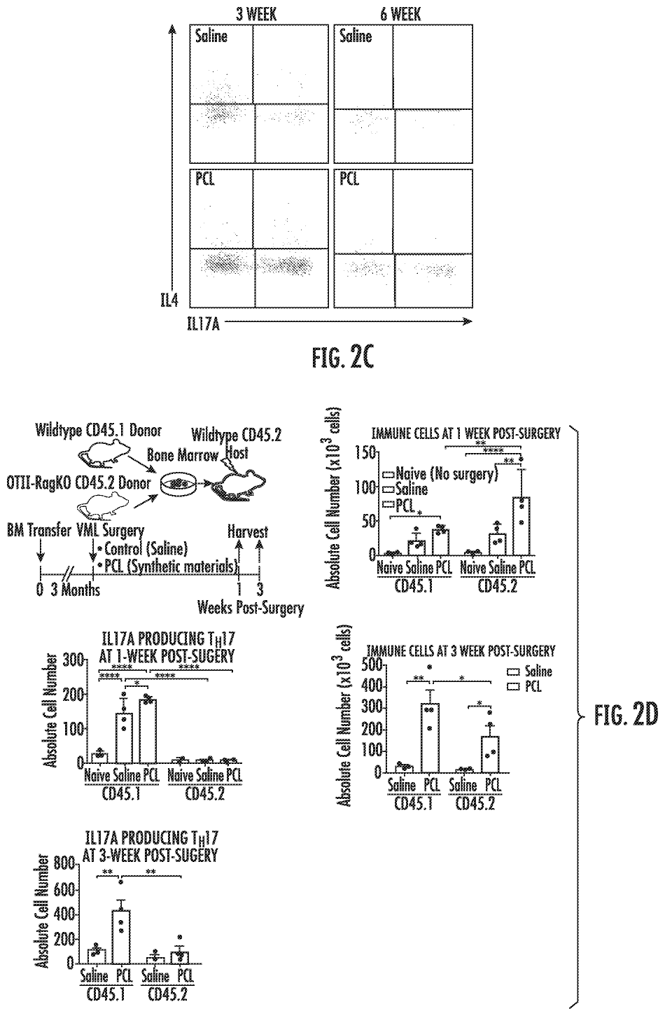

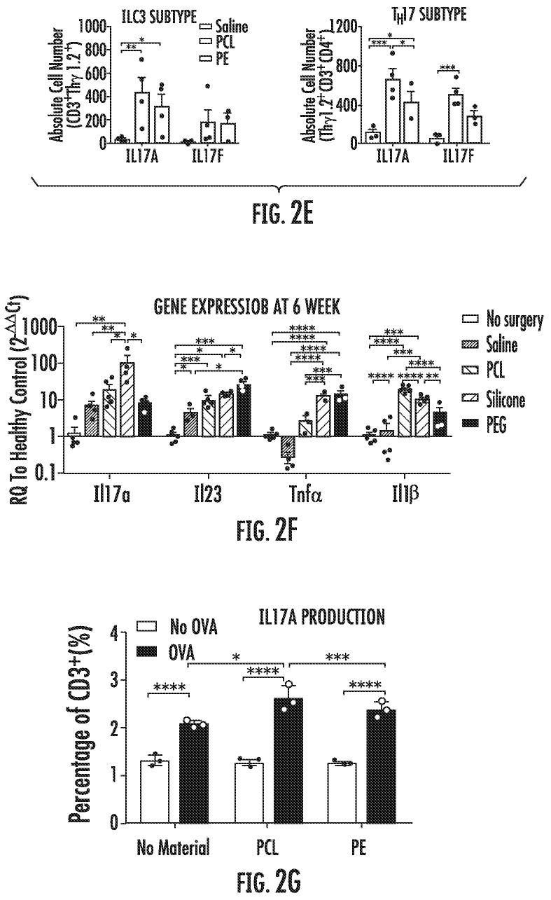

Implantation of PCL in surgical dermal and muscle wounds increased IL17 expression in the tissue compared to saline controls. We identified three primary cell sources of IL17 that evolved over time: IL17 producing group 3 innate lymphoid cells (ILC3s; CD45+CD3-Thy1.2+) that can respond quickly to danger associated molecular pattern (DAMPs) and cytokines independently of TCR signaling, IL17-producing γδ+ T cells (γδ T17) that can recognize non- peptide antigens such as lipids, phosphoantigens and carbohydrates, and adaptive CD4+ T cells (TH17) that can be activated, specifically through the alpha beta TCR receptor (FIG. 2A-C) (33).

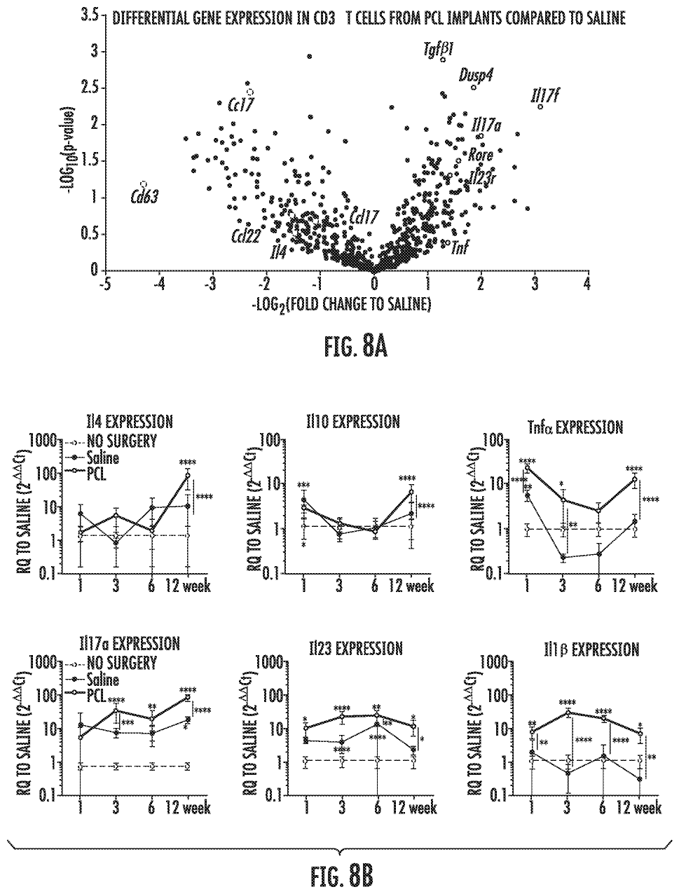

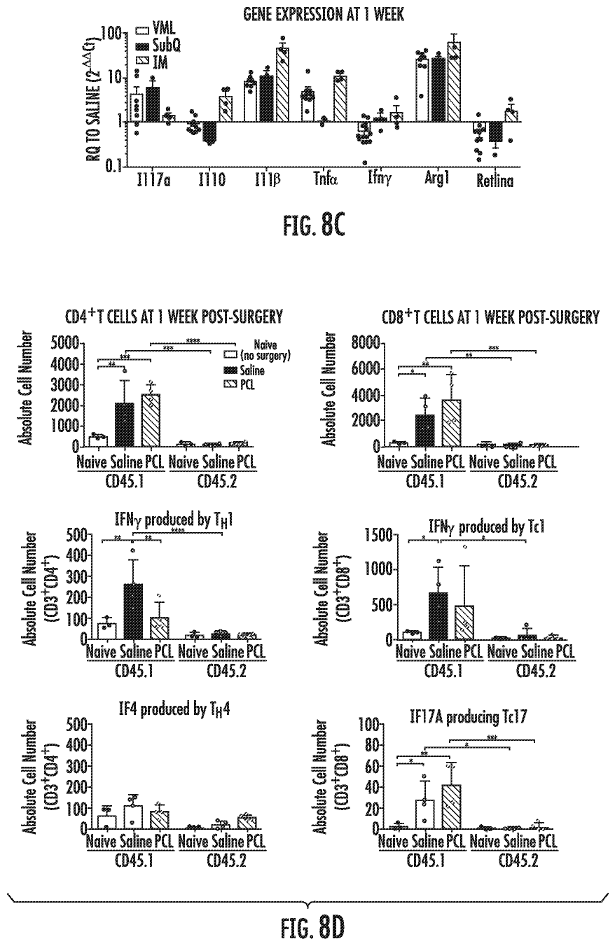

At 1-week post-implantation in the murine model, ILC3s and γδ+ T17 cells were the primary source of IL17 (FIG. 2B). CD4+ T cells exhibited no differences between IFNγ, IL4 or IL17 expression at one week by intracellular staining (FIG. 2A-C, FIG. 7B-C). After 3 and 6 weeks, IL17 expression shifted from ILC3s and γδ T17 cells to T_{H}17 cells. Analysis of the immune response to PCL in IL17A-GFP reporter mice validated that CD4+ T cells are the primary source of IL17A and there was minimal IL17A expression in myeloid cells at 3-weeks post-surgery (FIG. 7D). At 3-weeks post-surgery, CD8+ T cell response resolved whereas CD4+ T cells remained and continued to produce IL17 (FIG. 7E). Both IL17A and IL17F expression significantly increased in T cells (CD3+CD11b−) sorted from PCL-associated tissue compared to saline control at 1-week post-injury (p<0.0001) (FIG. 8A). Expression of Rorc (encoding the TH17 transcription factor RORγt), Il23r, and Dusp4, also increased in biomaterial-associated T cells, further supporting the TH17 immune response. Finally, gene expression analysis of the homogenized muscle tissue confirmed upregulation of Il17a expression in response to PCL implants compared to no implants over time (saline controls) (FIG. 8B). Expression of additional pro-inflammatory cytokines Il1β, tumor necrosis factor alpha (Tnfα), and Il23p19 also increased in the tissue (FIG. 8B). These pro-inflammatory cytokines drive immune activation and chronic inflammation through the differentiation and activation of TH17 cells (34).

While the degree and nature of the T_{H}17 and type 17 immune response varied depending on the biomaterial composition and physical properties, a range of synthetic materials with varying chemistry and physical properties activated this pathway in different tissue environments. Flow cytometry and gene expression analysis confirmed the T_{H}17 response to multiple biomaterial types over 6 weeks of implantation. At 3 weeks, flow cytometry showed expression of both IL17A and IL17F in CD4⁺ T cells and ILC3s in response to PCL and PE, with most cells expressing both forms of the protein (FIG. 2D). At 6 weeks, gene expression of type 17 immunity-associated genes Il1β, Tnfα, Il23, and Il17a increased in response to PCL, silicone, and PEG (FIG. 2E). PCL implanted into the subcutaneous space with less tissue disruption or injected directly into muscle without a large wound also activated a type 17 response (FIG. 8C). These results, combined with the clinical data from breast implants, suggest that the T_{H}17 response is activated in response to implants of various size, shape, and texture in different tissue environments.

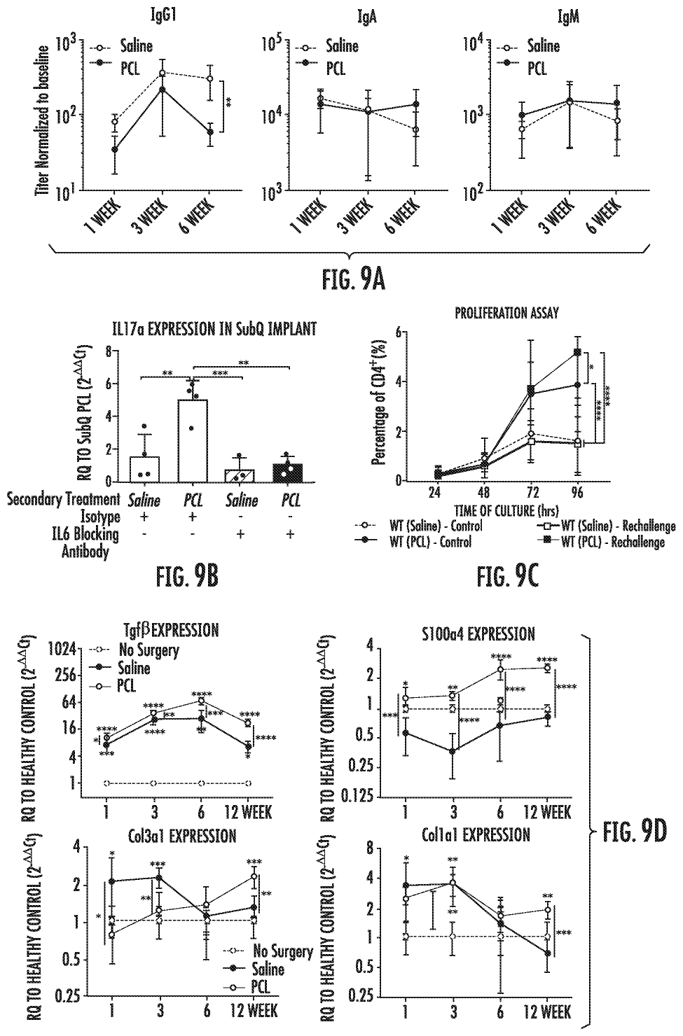

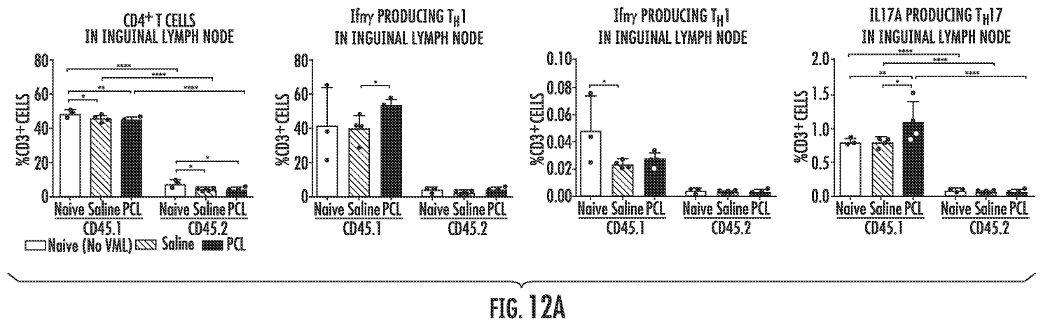

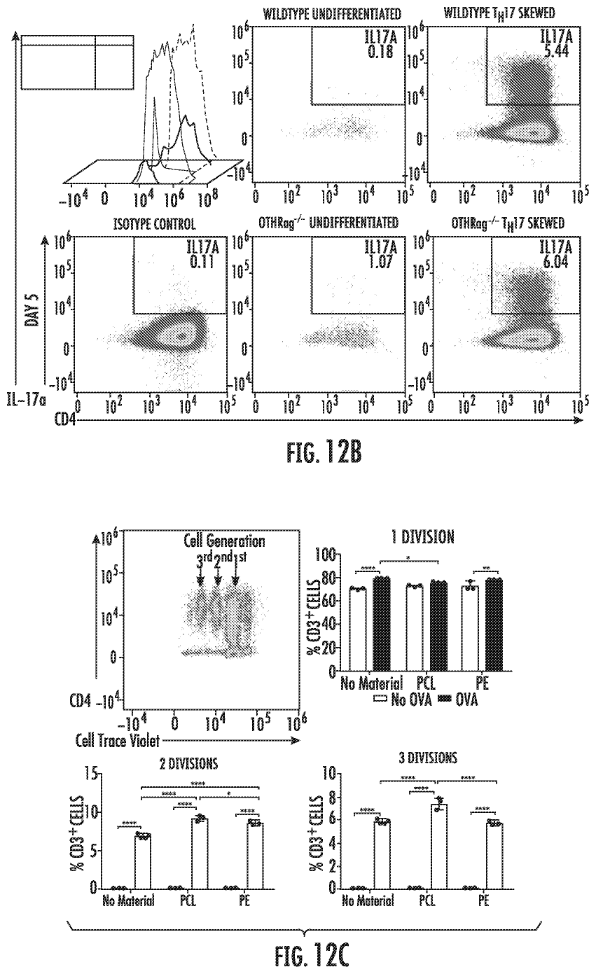

To determine whether the T_{H}17 response was mediated by the TCR, we used wildtype CD45.1 and OTII−Rag−/− CD45.2 bone marrow chimera mice (FIG. 2F). The OTII TCR transgenic Rag−/− CD4+ T cells are specific of ovalbumin (OVA) so we can test if there is non-specific activation of OVA-specific T cells in the context of a highly diverse TCR repertoire of the CD45.1 cells in the wound environment with or without PCL implantation. We found that CD45.1+ and CD45.2+ immune cells from donors responded to PCL implantation after 1 week. In the tissue and draining lymph nodes CD4+ T cells were primarily from the wild type (WT) CD45.1 donor (FIG. 8D, FIG. 12A). IL17 was expressed in wildtype CD45.1 CD4+ T cells in response to PCL implantation but was absent in the CD45.2 OTII-Rag−/− T cells. The capacity of OTII-Rag−/− T cells to undergo TH17 differentiation was confirmed in vitro, so the failure to produce IL17 in response to synthetic implants did not represent an intrinsic defect in their ability to undergo differentiation to TH17 (FIG. 12B). This suggests that the IL17 production by CD4+ T cells in response to PCL implantation was antigen specific. As previously described, biomaterials can modulate the T cell response to antigens thereby acting as an adjuvant. Splenocytes from OTII mice cultured in vitro with OVA in combination with PCL or PE for 48 hours showed increased T cell proliferation and cytokine production compared to without biomaterials (FIG. 2G, FIG. 12C). At 6-weeks post-surgery, we detected elevated IgG1 in the serum of mice implanted with PCL compared to saline controls, highlighting a B cell response and class switching with implant (FIG. 9A).

Since evidence supported engagement of the adaptive immune system in the FBR, we evaluated if there was a memory response to implant exposure. Mice were implanted with PCL and after one week were re-challenged with PCL in a subcutaneous implant. Two weeks afterPCL implantation in a VML wound and one week after addition of the subcutaneous implant, expression of Il17a in the second implant was evaluated. Previous exposure to PCL implants (PCL primed) enhanced Il1 7a expression in the subcutaneous implants compared to implants from mice that did not receive a previous PCL implant (not primed, saline treated wounds that did not receive PCL implants) (FIG. 9B). Treatment with IL6 neutralizing antibodies completely mitigated the increase in IL17A in the subcutaneous implant regardless of priming status.

We further assessed whether there is a PCL antigen specific response capable of inducing immunological memory using a combination of in vivo and in vitro experiments. Splenocytes were harvested from mice treated with PCL or saline-treated controls for 3 weeks. After labeling with a proliferation dye, we (re)challenged the cells with PCL in the culture. Cells derived from mice implanted with PCL had significantly higher proliferation at 96 hours compared to cells from mice that did not receive were not primed or no rechallenge control (FIG. 9C).

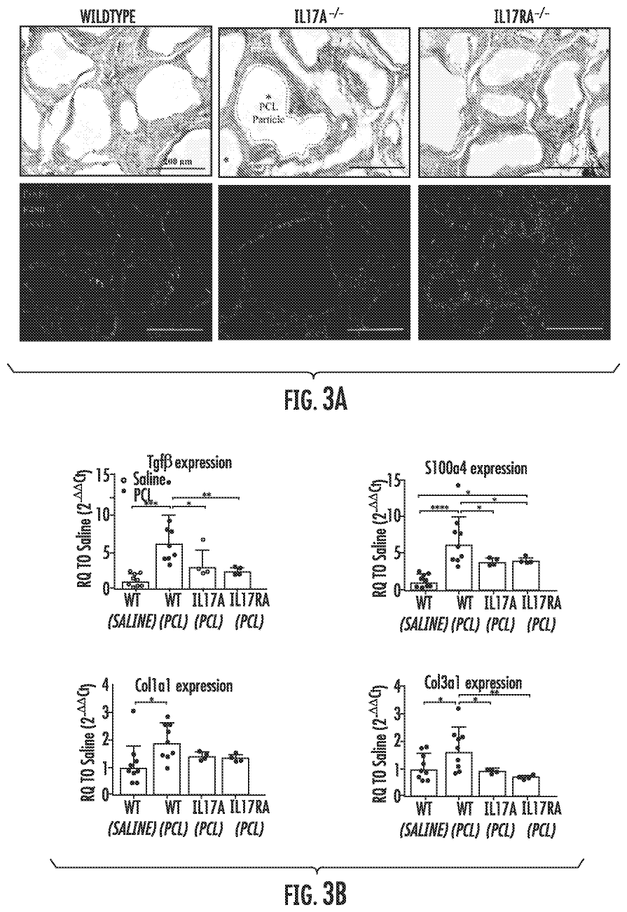

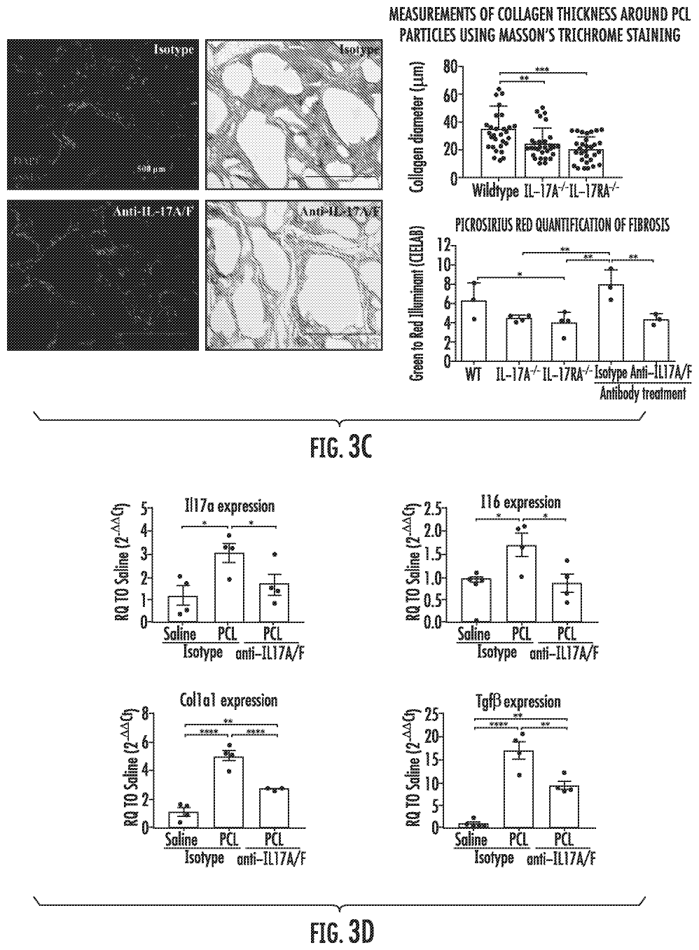

Chronic IL-17 in response to synthetic implants induces excess fibrosis: TH17 immune responses are implicated in fibrotic diseases in multiple tissue types including skin, heart, lung, and liver, though have not been studied in the context of FBR (35- 38). In the WT mice that demonstrated a type 17 immune response, the expression of fibrosis related genes after surgery evolved over time (FIG. 9D). Without a biomaterial, expression of the extracellular matrix genes collagen I and III initially increased after surgery then decreased by 6 weeks as normal healing progressed and tissue repaired. The presence of the PCL implant increased expression of collagen III and the fibrosis gene S100a4 after 6 and 12 weeks. Masson's trichrome and immunohistochemistry confirmed increased collagenous extracellular matrix and alpha smooth muscle actin protein (αSMA) surrounding the PCL particles (FIG. 3A), features typical of the FBR and fibrotic capsule formation.

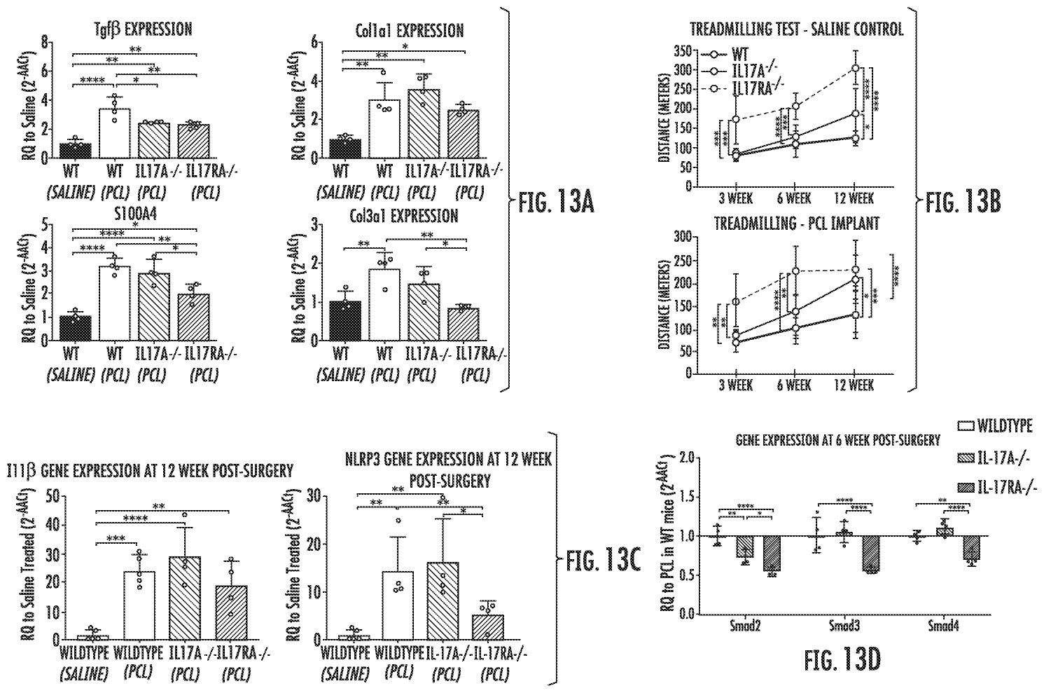

To evaluate if fibrosis associated with the FBR to biomaterials was IL17 dependent, we compared the implant response in IL17A−/− and IL17RA−/− mice with WT mice. We found that expression of fibrosis-related genes S100a4, Tgfβ, collagen I and III decreased in both knockout mice implanted with PCL compared to the WT mice (FIG. 3B, FIG. 13A). The quantity, composition, and organization of ECM visible around the PCL particles decreased in the knockout animals (FIG. 3A). In addition, αSMA immunofluorescence was absent in Il17 signaling deficient mice. Picrosirius red (PSR) visualized with a polarized lens produces birefringence that is specific for collagen fiber organization; larger collagen fibers are bright orange to red and thinner fibrils are green to yellow (39). A reduction in collagenous matrix and thinner fibrotic capsules around the PCL particles was confirmed in knockout mice compared to WT mice at 12-weeks post-surgery using image analysis and quantification of collagen thickness and green to red illuminant on PSR images (FIG. 3C). Functional recovery of the tissue repair was not impaired in the IL17A−/− and IL17RA−/− mice as assessed by treadmill testing (FIG. 13B). In addition, expression of the inflammasome associated gene Nlrp3 and IL1β reduced in PCL-implanted tissue in IL17RA−/− mice, but not in IL17A−/−, compared to the WT mice (FIG. 13C). We also found decreased Smad 2, Smad 3, and Smad 4 gene expression in IL17RA−/− mice compared to WT mice that was not observed in the IL17A−/− mice (FIG. 13D). These findings further support the IL17 dependent nature of the FBR to biomaterials.

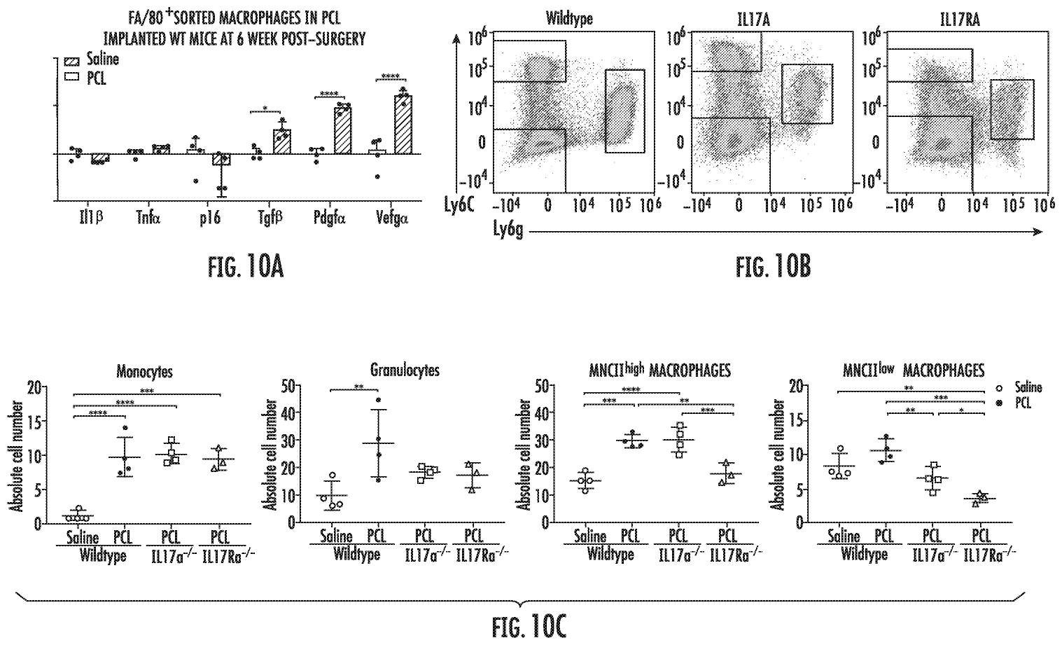

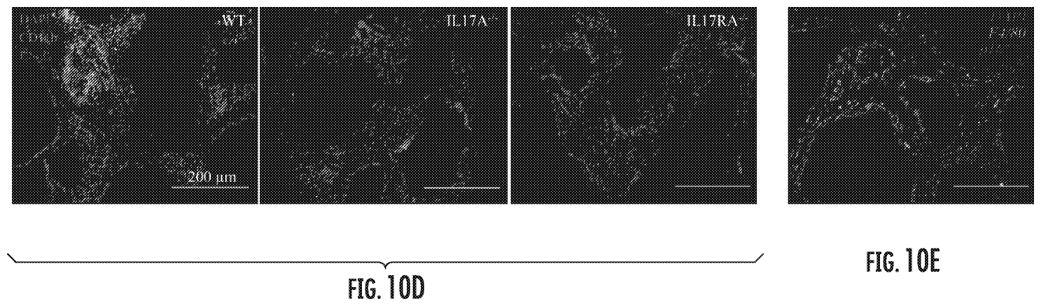

We next assessed the myeloid response associated with PCL implantation in the IL17 knockout mice. The number of neutrophils and macrophages increased in tissue implanted with synthetic materials. Macrophages sorted from the tissue implanted with PCL expressed higher profibrotic stimulating factors that directly activate fibroblasts including Tgfβ, platelet derived growth factor a (Pdgfα), and vascular endothelial growth factor α (Vegfα) (FIG. 10A). While the histocompatibility complex class II MHCIIhigh and MHCIIlow macrophages was altered. MHCIIhigh macrophage numbers were similar to the WT controls without implants in the IL17RA−/− mice while MHCIIlow macrophages significantly decreased in both IL17A−/− and IL17RA−/− mice (p<0.005). Neutrophils (CD11b+Ly6c+Ly6g+) and macrophages (CD11b+F4/80+), decreased in the IL17A−/− and IL17RA−/− mice compared to WT after 12 weeks (FIG. 10B-D). Flow cytometry and immunofluorescence confirmed fewer neutrophils in the IL17A−/− and IL17RA−/− mice implanted with PCL.

Results from the knockout studies suggest that blocking of IL17 signaling may reduce FBR-associated fibrosis without negatively impacting healing. We therefor sought to determine the therapeutic potential of blocking IL17 on fibrosis. Since IL17A and IL17F were both produced in response to the implant and the IL17RA−/− mice showed the greatest reduction in fibrosis compared to IL17A−/−, we evaluated delivery of IL17A and IL17F specific neutralizing antibodies (IL17A/F NAbs) to test the impact of functional neutralization of IL17R signaling on fibrosis. Mice were treated with IL17A/F NAbs 4 weeks after biomaterial implantation every other day for one week for a total of 5 injections. One week after the IL17A/F NAb treatment, IL17 and IL6 expression decreased. Expression of fibrosis-associated genes Tgfβ and Collagen I also decreased (FIG. 3D). Immunofluorescence of αSMA also decreased, further suggesting reduced fibrosis. Histologically, collagen density and fiber organization decreased with anti- IL17A/F treatment compared to isotype controls (FIG. 3C). PSR staining and quantification demonstrated that IL17A/F NAbs modulated ECM organization and reduced fibrosis that were similar to the knockout animals.

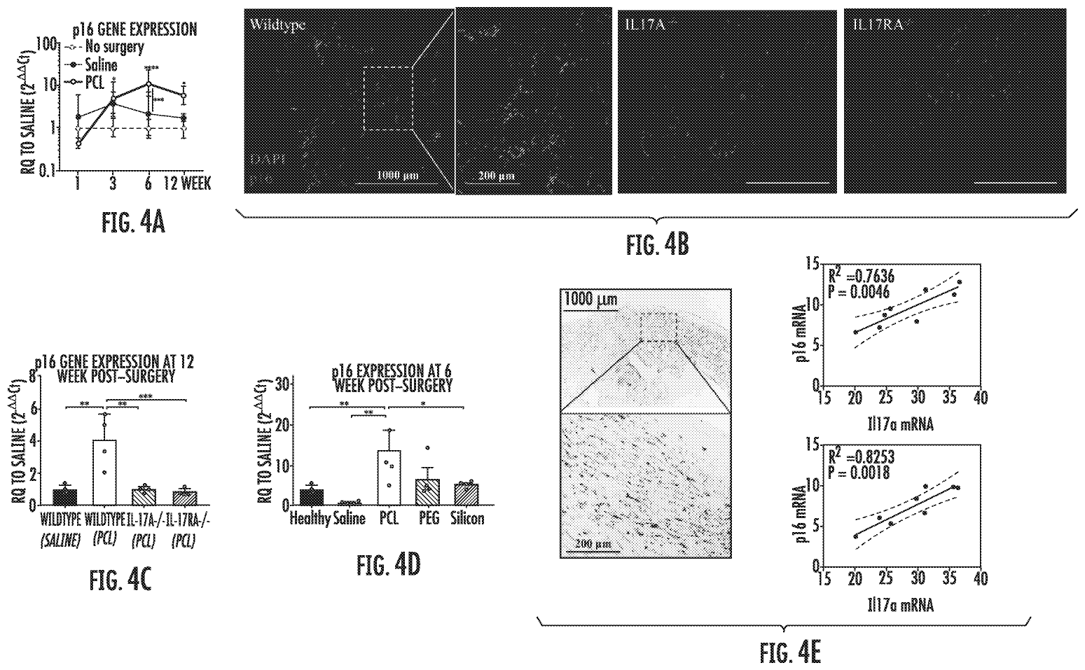

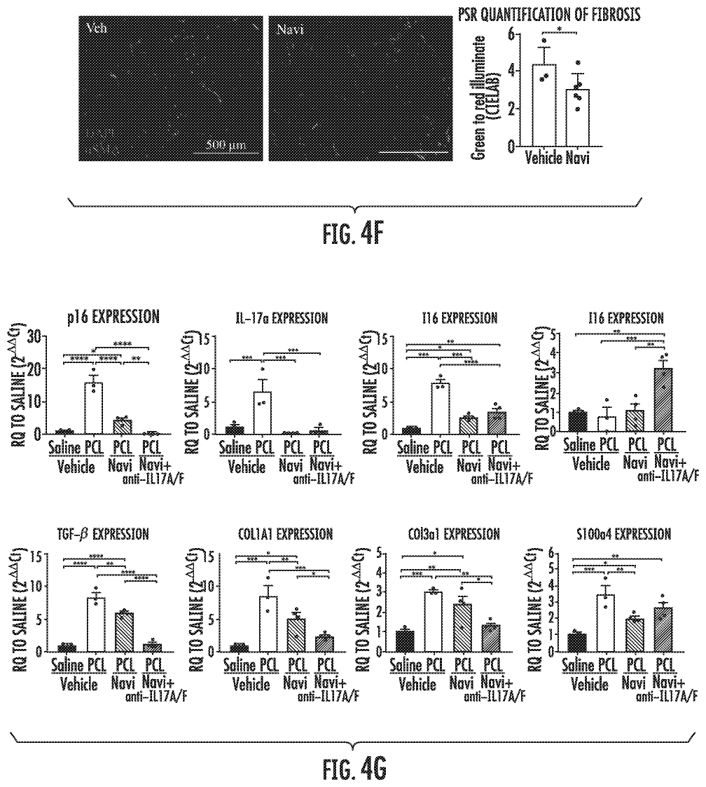

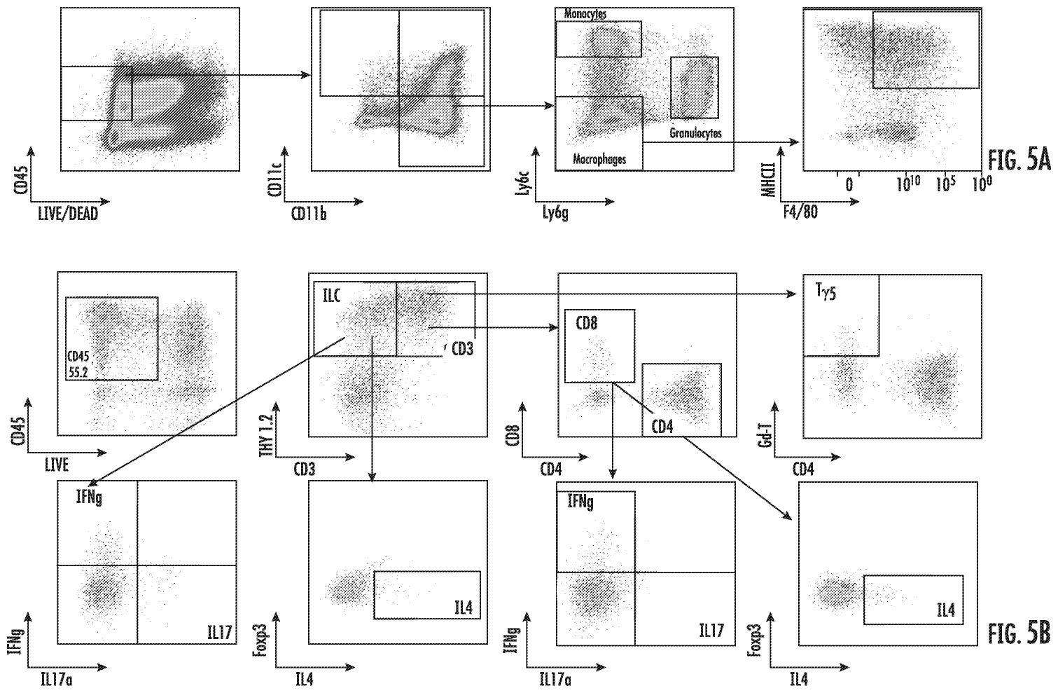

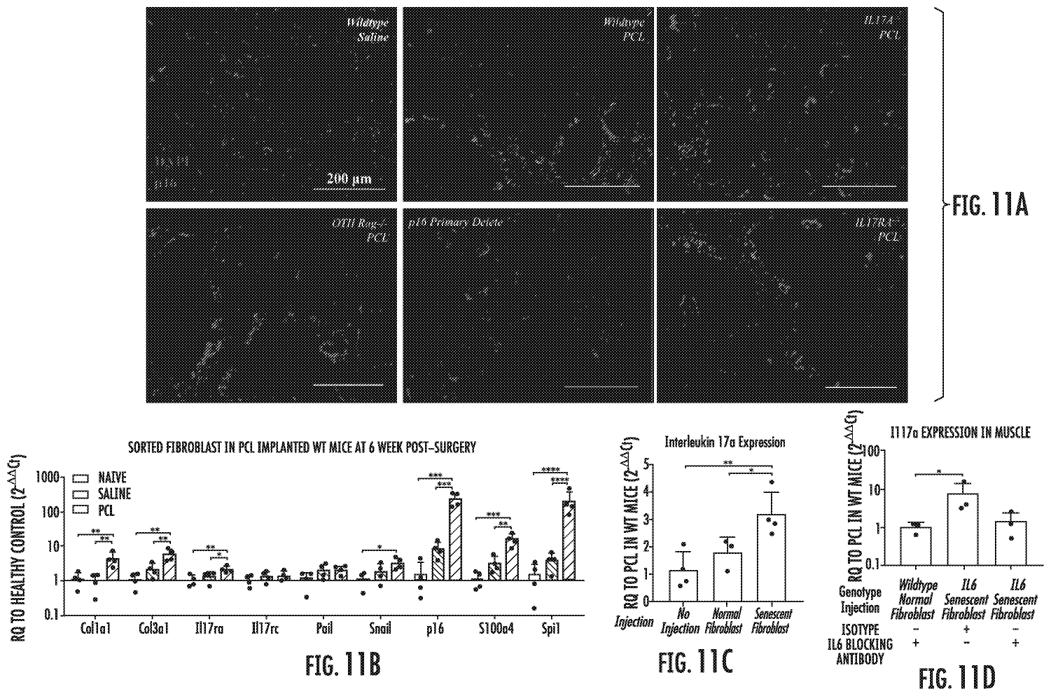

P16^{INK4a} senescent cells develop during the FBR and senolysis reduces fibrosis: IL6 is a critical mediator contributing to the differentiation of TH17 cells. Since IL6 is associated with the SASP produced by senescent cells, we investigated whether SnCs were a potential source of IL6 contributing to the chronic IL17 production and fibrosis (18). SnCs are characterized by expression of p16INK4a, p21 and SASP factors (40-42). Consistent with the kinetics of fibrosis development, p1 6INK4a expression significantly increased from 6 to 12 weeks after PCL implantation compared to saline controls (p<0.0001 at 6 weeks and p<0.01 at 12 weeks) (FIG. 4A). p1 6INK4a positive cells were localized to the peri-implant region and exhibited a fibroblastic morphology with a single nucleus (FIG. 4B, FIG. 10E, FIG. 11A). Fibroblasts sorted from PCL implants expressed significantly higher p16INK4a compared with healthy and saline controls, whereas sorted macrophages did not express p16INK4a (p<0.001) (FIG. 10A, FIG. 11B). SnC development was abrogated in both the IL17A−/− and IL17RA−/− mice where p16INK4a expression in PCL-implanted tissue was similar to the no implant controls at 12 weeks, demonstrating that SnC development depended on IL17 (FIG. 4C). All materials tested induced p16INK4a expression to varying degrees, suggesting that the response is material independent (FIG. 4D).

To further validate and define clinical relevance for the association of SnCs with fibrosis and the FBR, we evaluated patient tissue from breast implant exchanges. A large number of p16INK4a positive cells were present in the tissue surrounding the implants (FIG. 4E). Gene expression analysis of tissue surrounding the implants also showed a strong positive correlation between Il17 and p16INK4a (R2=0.7636, p=0.0046) in addition to Il17 and p21 (R2=0.8253, p=0.0018, FIG. 4E).

To further study the role of senescence in regulation of the FBR, we inject gamma-irradiated senescent fibroblasts locally with PCL implants in the VML model. Addition of that received normal fibroblasts with PCL (FIG. 11C). To determine the role of IL6 secreted by the transplanted senescent cells, we injected senescent fibroblasts into local injury of the IL6−/− mice who had been treated with PCL implants. In this model, the source of IL6 is limited to the injected senescent cells and not host senescent cells that develop around the implant. Mice that received senescent cells along with the implant significantly upregulated Il17a expression (FIG. 11D). However, treatment with IL6 neutralizing antibody diminished Il17a expression to baseline levels. This observation facilitates the notion that senescent cells were key drivers of IL17 response via their SASP, and IL6 in particular. To determine if clearance of SnCs associated with the FBR could reduce fibrosis, we administrated the senolytic agent Navitoclax (Navi, ABT-263) that selectively kills SnCs (25, 43). Navi was administered alone and in combination with anti-IL17A/F NAbs 4 weeks after implantation, when p16INK4a expression gradually increased with biomaterial implantation compared to control. Clearance of the SnCs reduced αSMA immunofluorescence, suggesting reduced fibrosis activity (FIG. 4F). One week after treatment (6 weeks after implantation), expression of p16INK4a significantly decreased confirming senolysis (p<0.0001) (FIG. 4G). Concomitant with p16INK4a and αSMA reduction, Il17a, Il16, Tgfβ, S100a4, collagen I and III gene expression decreased after senolytic treatment. These genes decreased even further when the senolytic was co-administered with anti-IL 17A/F NAbs (FIG. 4G). Altogether these findings demonstrate that cellular senescence sustains excess fibrosis and links to chronic IL17 and fibrogenesis during the foreign body response.

TABLE 1
List of Sybr Green Mouse mRNA Primers

| Primer | Forward Sequence | Primer | Reverse Sequence |
|---|---|---|---|
| β2m | CTC GGT GAC CCT GGT CTT TC | β2m | GGATTT CAA TGT GAG GCG GG |
| forward | | reverse | |
| Tnfα | GTC CAT TCC TGA GTT CTG | Tnfα | GAA AGG TCT GAA GGT AGG |
| forward | | reverse | |
| Il1β | GTA TGG GCT GGA CTG TTT C | Il1β | GCT GTC TGC TCA TTC ACG |
| forward | | reverse | |
| Retnla | CTT TCC TGA GAT TCT GCC CCA G | Retnla | CAC AAG CAC ACC CAG TAG CA |
| forward | | reverse | |
| Ifnγ | TCA AGT GGC ATA GAT GTG GAA | Ifnγ | TGA GGT AGA AAG AGA TAA TCT GG |
| forward | | reverse | |
| Il4 | ACA GGA GAA GGG ACG CCA T | Il4 | ACC TTG GAA GCC CTA CAG A |
| forward | | reverse | |
| Il17a | TCA GCG TGT CCA AAC ACT GAG | Il17a | CGC CAA GGG AGT TAA AGA CTT |
| forward | | reverse | |
| Arginase 1 | CAG AAG AAT GGA AGA GTC AG | Arginase 1 | CAG ATA TGC AGG GAG TCA CC |
| forward | | reverse | |
| Cdkn2a (p16) | AAT CTC CGC GAG GAA AGC | p16 | GTC TGC AGC GGA CTC CAT S |
| forward | | reverse | |
| Cdkn2b (p15) | AGA TCC CAA CGC CCT GAA C | p15 | CCC ATC ATC ATG ACC TGG ATT |
| forward | | reverse | |
| 110 | CAG GAC TTT AAG GGT TAC TTG GGT | Il10 | GCC TGG GGC ATC ACT TCT AC |
| forward | | reverse | |

TABLE 2
List of mouse TaqMan gene expression primers

| Primer | Assay ID: | Primer | Assay ID |
|---|---|---|---|
| β2m | Mm00437762 | Il17a | Mm00439618 |
| Col1a1 | Mm00801666 | Il23a | Mm00518984 |
| Col3a1 | Mm00802300 | S100a4 | Mm00803372 |
| Pai1 | Mm00435858 | Snai1 | Mm00441533 |
| Tgfβ-1 | Mm01178820 | p16 | Mm00494449 |
| Nlrp3 | Mm00840904 | Smad2 | Mm00487530 |
| Smad3 | Mm01170760 | Smad4 | Mm03023996 |

TABLE 3
List of human TaqMan gene expression primers

| Primer | Assay ID: | Primer | Assay ID |
|---|---|---|---|
| β2m | Hs00187842 | Il17a | Hs00174383 |
| Col1a1 | Hs00164004 | STAT3 | Hs00374280 |
| Col3a1 | Hs00943809 | S100a4 | Hs00243202 |
| p16 | Hs00923894 | Tgfβ-1 | Hs00998133 |
| Cdkn1a (p21) | Hs00355782 | Ifnγ | Hs00989291 |
| Il4 | Hs00174122 | Il6 | Hs00174131 |

TABLE 4
Antibodies used for flow cytometry (myeloid panel)

| Fluorophore | Marker | Manufacturer | Catalogue |
|---|---|---|---|
| Fixable Yellow | Live/Dead | Life Technologies | L34968 |
| AF488 | MHC-II | BioLegend | 107615 |
| PerCP-Cy5.5 | CD11b | BioLegend | 101227 |
| APC-Cy7 | CD11c | BioLegend | 117323 |
| PE-CF594 | Siglec F | BDbiosciences | 562757 |
| Pacific Blue | Ly6c | BioLegend | 128013 |
| AF647 | Ly6g | BioLegend | 127609 |
| BV510 | CD45 | BioLegend | 103137 |
| PE-Cy7 | F4/80 | BioLegend | 123113 |

TABLE 5
Antibodies used for flow cytometry (lymphoid panel)

| Fluorophore | Marker | Manufacturer | Catalogue |
|---|---|---|---|
| Fixable eFlour 780 | Live/Dead | Life Technologies | 65-0865-14 |
| BV786 | CD3 | BDbiosciences | 564010 |
| FITC | CD4 | BioLegend | 100509 |
| BV711 | CD8 | BioLegend | 100747 |
| PE-CF594 | γδ-TCR | BDbioscienes | 563532 |
| APC | IFNγ | BioLegend | 505809 |
| PE | IL4 | BioLegend | 504103 |
| AF700 | IL17A | BioLegend | 506914 |
| BV510 | CD45 | BioLegend | 103137 |
| PE-Cy7 | Thy1.2 | BioLegend | 140309 |

### Discussion

Medical implants derived from synthetic materials suffer to some extent from the FBR that not only impairs implant function and longevity but may also in turn impact the host. There are numerous anecdotal reports of systemic illnesses associated with orthopedic and soft tissueimplants most notably the breast implant syndrome (44, 45). With only local inflammatory components considered relevant to the FBR, direct correlation of implant responses with systemic immune pathologies was difficult to connect. Preclinical and clinical observations noted the presence of T cells with implants but their relevance to the fibrotic inflammatory response was unclear (14, 46). Here we present evidence of an IL17 inflammatory response and cellular senescence in the FBR to biomaterials and implicate them in the associated fibrotic response.

T cells are increasingly recognized for their role in determining repair pathways after tissue damage. We observed a TH17 response to biomaterials with multiple implant chemistries in numerous anatomical locations. B cells and their antibody production have already been associated with tissue damage and more recently with synthetic materials (47-49). For example, a B cell response to synthetic materials was discovered with implantation in the intraperitoneal cavity. Moreover, the response was independent of material chemistry and physical properties (49). In our studies, we utilized particles of materials in order to fill tissue defects in the murine model. These particulates had different sizes, textures and surface properties and can themselves activate a rapid, inflammasome-mediated innate immune responses (50). The presence of T_{H}17 cells in all of clinical samples tested, from large (non-particulate) and diverse implant surfaces, suggests the IL17 pathway is conserved in biomaterial-associated fibrosis and is broadly relevant and clinically applicable. Mechanical forces created by the implant and the related mechanotransduction of those signals can regulate cellular behavior (51). Biomaterial properties such as stiffness, size, and surface topography can influence cell behavior and cytokine production (52-54). Mechanical strain can modulate fibroblast proliferation and gene expression and may therefore be an important factor in promoting IL17 and senescence in the FBR. Further studies may elucidate unique aspects of the IL17 and immune response with different materials.

The induction of type 17 inflammation has been implicated in tissue fibrosis. For example, IL17 is a central contributor to pathogenic lung and liver fibrosis (55, 56). While TH17 responses are a mechanism to combat extracellular pathogens, they are also associated with autoimmune diseases. For example, TH17 cells are implicated in rheumatoid arthritis and Sjogren's syndrome and therapies inhibiting IL17 ameliorate disease symptoms. Induction of a T_{H}17 immune response to biomaterials is supported by the chronic neutrophil response to synthetic materials since IL17 induces chronic neutrophilia. Multiple studies have now demonstrated a negligible impact of neutrophil depletion on fibrosis around biomaterials, suggesting that they are not inducing fibrosis but a byproduct (49, 57).

The discovery of senescent cells in the FBR presents a unique mechanism for the sustained type 17 inflammation and fibrosis around implants in addition to a new therapeutic target.

Senolytic compounds are being developed to treat numerous age-related diseases including arthritis, cardiovascular and Alzheimer's disease (58, 59). Clinical studies testing senolytics in idiopathic pulmonary fibrosis are already showing efficacy and more clinical studies are ongoing (60, 61). The SASP secreted by SnCs include cytokines associated with a T_{H}17 immune response including IL6, IL1β, and the loss of SnCs in the IL17 knockout models further supports the IL17-SnC connection. It is possible the TH17 response is being driven locally by material auto-antigens or antigens, possibly facilitated by the senescent cells, as appears to be the case in post-traumatic osteoarthritis (62). The morphology of the p16INK4a positive cells appeared fibroblastic and sorted fibroblasts expressed significantly higher p16INK4a suggesting that stromal fibroblasts are an important source of senescence in the FBR. Other, not yet unidentified cell types, may become senescent during the FBR and further studies utilizing new genetic tools will help elucidate the source and phenotype of SnCs along with possible connections to myofibroblasts.

Canonical activation of T cells requires presentation of antigen and costimulatory factors. The lack of CD4+ T cell response to PCL in the chimeric OTII−Rag−/− T cells confirms that the TH17 implant response is antigen dependent. One explanation of an antigen-specific T cell response to materials that do not contain protein is a low-level chemical derivatization self- protein by components of the synthetic material, such that they now appear as “non-self”. The finding of IgG in response to implantation of a synthetic material supports this hypothesis, since haptenization of proteins is necessary for T cell help for Ig switching in activated hapten-specific B cells (63, 64). Combinations of a biomaterial with host proteins can activate T cells as in the case of titanium implant debris that creates a metal-protein complex that can elicit cell responses (65). Addition of an implant in the context of tissue damage may modulate the natural adaptive damage response by modifying self-antigens or impacting antigen presentation. Finally, there is evidence that T cells can specifically respond to non-peptidic repeating structures such as sugars and lipids and thus may recognize repeating polymer structures (66, 67). Regardless of the antigen or combination of antigens, the type 17 immune response is activated and directing key aspects of the FBR and targeting this pathway has therapeutic benefits for biocompatibility.

Engagement of the adaptive immune system introduces the potential influence of environment factors in the FBR beyond the standard genetic that can modulate adaptive immune responses such as infection, history of antigen exposure, and the microbiome. The murine models used to expand upon the findings from patient samples cannot fully recapitulate the impact of human aging, additional medical conditions or comorbidities, and anatomical placement on the clinical response. In addition, the specific antigens that lead to the activation and differentiation of CD4⁺ T cells toward type 17 adaptive immunity remain to be elucidated. Future studies will investigate the impact of the multiple immunomodulatory environmental factors that may impact TH17 and senescence responses to biomaterial implants.

### REFERENCES

1. J. M. Anderson, Inflammatory response to implants. ASAIO Trans 34, 101-107 (1988).
2. J. Singh, H. Inaty, S. Mukhopadhyay, A. C. Mehta, Chronic Pulmonary Silicone Embolism from Breast Augmentation Is Not a Common Finding in Explanted Lungs. Pulm Med 2018, 2987072-2987072 (2018).
3. C. J. Coroneos, J. C. Selber, A. C. Offodile, C. E. Butler, M. W. Clemens, US FDA breast implant postapproval studies: long-term outcomes in 99,993 patients. Annals of surgery 269, 30-36 (2019).
4. A. M. Hart, M. J. Lechowicz, K. K. Peters, J. Holden, G. W. Carlson, Breast Implant—Associated Anaplastic Large Cell Lymphoma: Report of 2 Cases and Review of the Literature. Aesthetic Surgery Journal 34, 884-894 (2014).
5. V. W. Chen, D. Hoang, S. Clancy, Breast Implant-Associated Bilateral B-Cell Lymphoma. Aesthetic Surgery Journal, sjy093-sjy093 (2018).
6. N. Shahriari, K. Ferenczi, P. W. Heald, Breast implant-associated anaplastic large cell lymphoma: A review and assessment of cutaneous manifestations. International Journal of Women's Dermatology 3, 140-144 (2017).
7. R. Marchant et al., In vivo biocompatibility studies. I. The cage implant system and a biodegradable hydrogel. J Biomed Mater Res 17, 301-325 (1983).
8. J. M. Anderson, A. Rodriguez, D. T. Chang, Foreign body reaction to biomaterials. Seminars in immunology 20, 86-100 (2008).
9. B. D. Ratner, Surface characterization of biomaterials by electron spectroscopy for chemical analysis. Annals of biomedical engineering 11, 313-336 (1983).
10. J. M. Anderson, A. K. McNally, Biocompatibility of implants: lymphocyte/macrophage interactions. Semin Immunopathol 33, 221-233 (2011).
11. G. S. Baht, L. Vi, B. A. Alman, The Role of the Immune Cells in Fracture Healing. Curr Osteoporos Rep 16, 138-145 (2018).
12. W. Liao, T. K. Hei, S. K. Cheng, Radiation-Induced Dermatitis is Mediated by IL17-Expressing γδ T Cells. Radiation research 187, 454-464 (2017).
13. K. Sadtler et al., Developing a pro-regenerative biomaterial scaffold microenvironment requires T helper 2 cells. 352, 366-370 (2016).
14. D. Wolfram et al., T regulatory cells and T_{H}17 cells in peri-silicone implant capsular fibrosis. Plast Reconstr Surg 129, 327e-337e (2012).
15. K. M. Hotchkiss, N. M. Clark, R. Olivares-Navarrete, Macrophage response to hydrophilic biomaterials regulates MSC recruitment and T-helper cell populations. Biomaterials 182, 202-215 (2018).
16. R. P. Garay, R. El-Gewely, J. K. Armstrong, G. Garratty, P. Richette, Antibodies against polyethylene glycol in healthy subjects and in patients treated with PEG-conjugated agents. Expert Opinion on Drug Delivery 9, 1319-1323 (2012).
17. M. S. Wilson et al., Bleomycin and IL-1βmediated pulmonary fibrosis is IL-17A dependent. 207, 535-552 (2010).
18. B. G. Childs, M. Durik, D. J. Baker, J. M. van Deursen, Cellular senescence in aging and age-related disease: from mechanisms to therapy. Nature medicine 21, 1424-1435 (2015).
19. B. Ritschka et al., The senescence-associated secretory phenotype induces cellular plasticity and tissue regeneration. Genes & development 31, 172-183 (2017).
20. S. Watanabe, S. Kawamoto, N. Ohtani, E. Hara, Impact of senescence-associated secretory phenotype and its potential as a therapeutic target for senescence-associated diseases. Cancer science 108, 563-569 (2017).
21. M. J. Schafer et al., Cellular senescence mediates fibrotic pulmonary disease. Nature Communications 8, 14532 (2017).
22. P. Chalan, A. van den Berg, B.-J. Kroesen, L. Brouwer, A. Boots, Rheumatoid Arthritis,

Immunosenescence and the Hallmarks of Aging. Current Aging Science 8, 131-146 (2015).
23. A. K. Palmer et al., Cellular Senescence in Type 2 Diabetes: A Therapeutic Opportunity. Diabetes 64, 2289 (2015).
24. G. Katsuumi, I. Shimizu, Y. Yoshida, T. Minamino, Vascular Senescence in Cardiovascular and Metabolic Diseases. Frontiers in cardiovascular medicine 5, 18-18 (2018).
25. D. Lagares et al., Targeted apoptosis of myofibroblasts with the BH3 mimetic ABT-263 reverses established fibrosis. Science translational medicine 9, eaal3765 (2017).
26. Q. Li et al., Age-associated alteration in Th17 cell response is related to endothelial cell senescence and atherosclerotic cerebral infarction. American journal of translational research 9, 5160-5168 (2017).
27. T. J. Harris et al., Cutting edge: An in vivo requirement for STAT3 signaling in T_{H}17 development and T_{H}17-dependent autoimmunity. The Journal of Immunology 179, 4313-4317 (2007).
28. F. Annunziato, C. Romagnani, S. J. J. o. A. Romagnani, C. Immunology, The 3 major types of innate and adaptive cell-mediated effector immunity. 135, 626-635 (2015).

29. B. D. Southern et al., in C78. FIBROSIS: MEDIATORS AND MODULATORS. (American Thoracic Society, 2017), pp. A6422-A6422.
30. X.-m. Meng, D. J. Nikolic-Paterson, H. Y. Lan, TGF-β: the master regulator of fibrosis. Nature Reviews Nephrology 12, 325 (2016).
31. C. Z. C. Chen, M. Raghunath, Focus on collagen: in vitro systems to study fibrogenesis and antifibrosis_state of the art. Fibrogenesis & Tissue Repair 2, 7 (2009).
32. 0. Veiseh et al., Size- and shape-dependent foreign body immune response to materials implanted in rodents and non-human primates. Nature materials 14, 643-651 (2015).
33. E. M. Beckman et al., Recognition of a lipid antigen by CD1-restricted αβ+ T cells. Nature 372, 691 (1994).
34. C. E. Sutton et al., Interleukin-1 and IL-23 Induce Innate IL-17 Production from γδ T Cells, Amplifying Th17 Responses and Autoimmunity. Immunity 31, 331-341 (2009).
35. A. Pellicoro, P. Ramachandran, J. P. Iredale, J. A. Fallowfield, Liver fibrosis and repair: immune regulation of wound healing in a solid organ. Nature Reviews Immunology 14, 181 (2014).
36. L. A. Borthwick, T. A. Wynn, A. J. Fisher, Cytokine mediated tissue fibrosis( ). Biochimica et biophysica acta 1832, 1049-1060 (2013).
37. L. Wu et al., Cardiac fibroblasts mediate IL-17A-driven inflammatory dilated cardiomyopathy. Journal of Experimental Medicine 211, 1449-1464 (2014).
38. S. Mi et al., Blocking IL-17A promotes the resolution of pulmonary inflammation and fibrosis via TGF-β1-dependent and -independent mechanisms. The Journal of Immunology 187, 3003-3014 (2011).
39. M. T. Wolf et al., Polypropylene surgical mesh coated with extracellular matrix mitigates the host foreign body response. Journal of Biomedical Materials Research Part A: An Official Journal of The Society for Biomaterials, The Japanese Society for Biomaterials, and The Australian Society for Biomaterials and the Korean Society for Biomaterials 102, 234-246 (2014).
40. G. Nelson et al., A senescent cell bystander effect: senescence-induced senescence. Aging cell 11, 345-349 (2012).
41. R. Salama, M. Sadaie, M. Hoare, M. Narita, Cellular senescence and its effector programs. Genes & development 28, 99-114 (2014).
42. G. H. Stein, L. F. Drullinger, A. Soulard, V. Dulić, Differential roles for cyclin-dependent kinase inhibitors p21 and p16 in the mechanisms of senescence and differentiation in human fibroblasts. Molecular and cellular biology 19, 2109-2117 (1999).
43. J. Chang et al., Clearance of senescent cells by ABT263 rejuvenates aged hematopoietic stem cells in mice. Nature medicine 22, 78-83 (2016).
44. M. M. Silva et al., Systemic inflammatory reaction after silicone breast implant. Aesthetic Plast Surg 35, 789-794 (2011).
45. J. Wawrzynski, J. A. Gil, A. D. Goodman, G. R. Waryasz, Hypersensitivity to Orthopedic Implants: A Review of the Literature. Rheumatol Ther 4, 45-56 (2017).
46. W. G. Brodbeck et al., In vivo leukocyte cytokine mRNA responses to biomaterials are dependent on surface chemistry. J Biomed Mater Res A 64, 320-329 (2003).
47. J. Chen, J. C. Crispin, T. F. Tedder, J. D. Lucca, G. C. Tsokos, B Cells Contribute to Ischemia/Reperfusion-Mediated Tissue Injury. Journal of autoimmunity 32, 195-200 (2009).
48. Z. Julier, A. J. Park, P. S. Briquez, M. M. Martino, Promoting tissue regeneration by modulating the immune system. Acta Biomaterialia 53, 13-28 (2017).
49. J. C. Doloff et al., Colony Stimulating Factor-1 Receptor is a central component of the foreign body response to biomaterial implants in rodents and non-human primates. Nature materials 16, 671-680 (2017).
50. I. M. Meraz et al., Activation of the inflammasome and enhanced migration of microparticle-stimulated dendritic cells to the draining lymph node. Molecular pharmaceutics 9, 2049-2062 (2012).
51. Y. Zhang et al., Progress in Integrative Biomaterial Systems to Approach Three-Dimensional Cell Mechanotransduction. Bioengineering (Basel) 4, 72 (2017).
52. M. C. Regier et al., The Influence of Biomaterials on Cytokine Production in 3D Cultures. Biomacromolecules 18, 709-718 (2017).
53. P. K. Kreeger, L. E. Strong, K. S. Masters, Engineering Approaches to Study Cellular

Decision Making. Annual review of biomedical engineering 20, 49-72 (2018).
54. D. Duscher et al., Mechanotransduction and fibrosis. J Biomech 47, 1997-2005 (2014).
55. E. Cipolla et al., IL-17A deficiency mitigates bleomycin-induced complement activation during lung fibrosis. FASEB journal: official publication of the Federation of American Societies for Experimental Biology 31, 5543-5556 (2017).
56. F. Meng et al., IL-17 signaling in inflammatory cells, Kupffer cells and Hepatic Stellate cells exacerbates liver fibrosis. Gastroenterology 143, 765-776.e761-763 (2012).
57. K. Sadtler et al., Divergent immune responses to synthetic and biological scaffolds. Biomaterials 192, 405-415 (2019).
58. M. Xu et al., Senolytics improve physical function and increase lifespan in old age. Nature Medicine 24, 1246-1256 (2018).
59. C. López-Otín, M. A. Blasco, L. Partridge, M. Serrano, G. Kroemer, The hallmarks of aging. Cell 153, 1194-1217 (2013).
60. J. L. Kirkland, T. Tchkonia, Cellular Senescence: A Translational Perspective. EBioMedicine 21, 21-28 (2017).
61. J. N. Justice et al., Senolytics in idiopathic pulmonary fibrosis: Results from a first-in-human, open-label, pilot study. EBioMedicine, (2019).
62. O. H. Jeon et al., Local clearance of senescent cells attenuates the development of post-traumatic osteoarthritis and creates a pro-regenerative environment. Nature Medicine 23, 775 (2017).
63. G. W. Siskind, W. E. Paul, B. Benacerraf, Studies on the effect of the carrier molecule on antihapten antibody synthesis. I. Effect of carrier on the nature of the antibody synthesized. The Journal of experimental medicine 123, 673-688 (1966).
64. H. M. Dintzis, R. Z. Dintzis, B. Vogelstein, Molecular determinants of immunogenicity:

the immunon model of immune response. Proceedings of the National Academy of Sciences of the United States of America 73, 3671-3675 (1976).
65. A. J. Mitchelson et al., Biomaterial Hypersensitivity: Is It Real? Supportive Evidence and Approach Considerations for Metal Allergic Patients following Total Knee Arthroplasty. BioMed Research International 2015, 137287 (2015).
66. P. Zhang, F. Sun, S. Liu, S. Jiang, Anti-PEG antibodies in the clinic: Current issues and beyond PEGylation. Journal of controlled release: official journal of the Controlled Release Society 244, 184-193 (2016).
67. M. Lepore, L. Mori, G. De Libero, The Conventional Nature of Non-MHC-Restricted T Cells. Frontiers in immunology 9, 1365-1365 (2018).
68. B. M. Sicari et al., A murine model of volumetric muscle loss and a regenerative medicine approach for tissue replacement. Tissue Eng Part A 18, 1941-1948 (2012).
69. K. J. Livak, T. D. Schmittgen, Analysis of Relative Gene Expression Data Using Real-Time Quantitative PCR and the 2-ΔΔCT Method. Methods 25, 402-408 (2001).

### Other Embodiments

From the foregoing description, it will be apparent that variations and modifications may be made to the invention described herein to adopt it to various usages and conditions. Such embodiments are also within the scope of the following claims.

All citations to sequences, patents and publications in this specification are herein incorporated by reference to the same extent as if each independent patent and publication was specifically and individually indicated to be incorporated by reference.

What is claimed:

1. A method of inhibiting a foreign body response (FBR) in a subject, comprising:
administering to the subject a pharmaceutical composition comprising a therapeutically effective amount of an agent which inhibits interleukin-17 (IL-17) activity or function, thereby, inhibiting the foreign body response.

2. The method of claim 1, wherein the agent inhibiting IL-17 inhibits IL-17-producing γδ T cells and CD4⁺ T_{H}17 cells in tissue surrounding the foreign body.

3. The method of claim 1, wherein administration of the agent inhibiting IL-17 results in reduction of expression of p16, p21, IL-17, type I collagen, S100a4 or combinations thereof.

4. The method of any one of claims 1 through 3, further comprising administering a senolytic agent, a senomorphic agent, an inhibitor of interleukin-6 (IL-6), an inhibitor of interleukin 1β (IL-1β), an inhibitor of tumor necrosis factor α (TNFα), an inhibitor of interleukin-23 (IL-23) or combinations thereof.

5. The method of claim 4, wherein the senolytic agent comprises: dasatinib, quercetin, ABT-263 (navitoclax), ABT-737, piperlongumine (PL), fisetin, HSP90 inhibitors, A1331852, A1155463, ATTAC, BCL-X_{L} inhibitor, analogues or combinations thereof.

6. The method of any one of claims 1 through 5, wherein the senolytic agent or the agent inhibiting IL-17 comprise: antibodies, antibody fragments, oligonucleotides, polynucleotides, antisense oligonucleotides, enzymes, gene editing agents, nucleases, peptides, polypeptides, small molecules, synthetic compounds, natural compounds or combinations thereof.

7. The method of claim 6, wherein the agent inhibiting IL-17 expression or function and the senolytic agent are administered concomitantly or at different times.

8. A method of inhibiting a T helper 17 (T_{H}17) cellular response in a subject, comprising:
administering to the subject a pharmaceutical composition comprising a therapeutically effective amount of an inhibitor of interleukin-17 (IL-17) activity or function, thereby,
inhibiting the T_{H}17 cellular response.

9. The method of claim 8, wherein the inhibitor of IL-17 inhibits expression of p16, p21, IL-17, type I collagen, S100a4 or combinations thereof.

10. The method of claim 8 or 9, further comprising administering a senolytic agent, an inhibitor of interleukin-6 (IL-6), an inhibitor of interleukin 1β (IL-1β) or combinations thereof.

11. The method of any one of claims 8 through 10, further comprising administering cytokines which reduce T_{H}17 cells, said cytokines comprise interleukin-4 (IL-4), interferon-gamma (IFN-γ), interleukin-12 (IL-12), (IL-27) or combinations thereof.

12. A composition comprising a therapeutically effective amount of an IL-17 inhibitory agent, a senolytic agent or a combination thereof.

13. The composition of claim 12, wherein the senolytic agent or the IL-17 inhibitory agent comprise: antibodies, antibody fragments, oligonucleotides, polynucleotides, antisense oligonucleotides, enzymes, gene editing agents, nucleases, peptides, polypeptides, small molecules, synthetic compounds, natural compounds or combinations thereof.

14. The composition of claim 12 or 13, wherein the senolytic agent comprises: dasatinib, quercetin, ABT-263 (navitoclax), ABT-737, piperlongumine (PL), fisetin, HSP90 inhibitors, A1331852, A1155463, ATTAC, BCL-X_{L} inhibitors, analogues or combinations thereof

15. The composition of any one of claims 12 through 14, further comprising cytokines, said cytokines comprising interleukin-4 (IL-4), interferon-gamma (IFN-γ), interleukin-12 (IL-12), (IL-27) or combinations thereof.

16. The composition of any one of claims 12 through 15, further comprising a senomorphic agent.

17. A composition comprising a therapeutically effective amount of an IL-17 inhibitory agent and a senolytic agent.

18. The composition of claim 17, wherein the senolytic agent or the IL-17 inhibitory agent comprise: antibodies, antibody fragments, oligonucleotides, polynucleotides, antisense oligonucleotides, enzymes, gene editing agents, nucleases, peptides, polypeptides, small molecules, synthetic compounds, natural compounds or combinations thereof.

19. The composition of claim 17, wherein the senolytic agent comprises: dasatinib, quercetin, ABT-263 (navitoclax), ABT-737, piperlongumine (PL), fisetin, HSP90 inhibitors, A1331852, A1155463, ATTAC, BCL-X_{L} inhibitors, analogues or combinations thereof .

20. The composition of any one of claims 17 through 19, further comprising cytokines, said cytokines comprising interleukin-4 (IL-4), interferon-gamma (IFN-γ), interleukin-12 (IL-12), (IL-27) or combinations thereof.

21. The composition of any one of claims 17 through 20, further comprising a senomorphic agent.
